(19) United States
(12) Patent Application Publication
(10) Pub. No.: US 2024/0358806 A1
(43) Pub. Date: Oct. 31, 2024

(54) METHODS FOR THE PREVENTION AND TREATMENT OF SYNUCLEINOPATHIES

(71) Applicant: Vaxxinity, Inc., Merritt Island, FL (US)

(72) Inventor: Jean-Cosme Dodart, Merritt Island, FL (US)

(21) Appl. No.: 18/688,196

(22) PCT Filed: Sep. 1, 2022
(86) PCT No.: PCT/US22/75836

§ 371 Date: Feb. 29, 2024

Related U.S. Application Data
(60) Provisional application No. 63/239,505, filed on Sep. 1, 2021.

(51) Int. Cl. A61K 39/00; A61P 25/16; A61P 25/28

(52) U.S. Cl. CPC A61K 39/0008; A61P 25/16; A61P 25/28; A61K 2039/55505; A61K 2039/55561; A61K 2039/575

(57) ABSTRACT

This disclosure provides methods and compositions for preventing and treating synucleinopathies.

### SEQUENCE LISTING

The instant application contains a Sequence Listing which has been submitted electronically in XML format and is hereby incorporated by reference in its entirety. Said XML copy, created on Aug. 31, 2022, is named 51615-002WO2_Sequence_Listing_8_31_22_ and is 272,021 bytes in size.

### FIELD OF THE INVENTION

This disclosure relates to methods for preventing and treating synucleinopathies.

### BACKGROUND OF THE INVENTION

Synucleinopathies are chronic progressive neurodegenerative diseases that are characterised by accumulation of alpha-synuclein (α-syn) in the brain. The synucleinopathies include Parkinson's disease (PD), Parkinson's disease dementia (PDD), dementia with Lewy bodies (DLB), and multiple system atrophy (MSA) (Outeiro et al., Mol. Neurodegener. 14(1):5, 2019). Several treatment options are available for these diseases, but they only provide symptomatic relief and do not directly target the underlying pathology. With the current aging population, PD and DLB cases are escalating, which highlights the urgent need for developing therapies that can prevent or delay the progression of neurodegeneration.

α-Syn is predominantly an intracellular protein. However, a number of studies have demonstrated that extracellular α-syn also plays a role in disease and is involved in the propagation of α-syn between neurons. α-Syn can be detected in cerebrospinal fluid (CSF), as well as in the interstitial fluid (ISF) of the brain parenchyma (Emmanouilidou et al., PLoS One 6(7):e22225, 2011). α-Syn is mainly secreted from cells by exocytosis (Lee et al., J. Neurosci. 25(25):6016-6024, 2005) or directly released into the extracellular space due to cell lysis and death. Internalisation of the protein by neighbouring cells results in the formation of protein aggregates, which could result in the propagation of the disease to anatomically connected brain regions (Danzer et al., Mol. Neurodegener. 7:42, 2012; Lee et al., J. Biol. Chem. 285:9262-9272, 2010). This mechanism has been demonstrated in cell culture studies in which addition of α-syn preformed fibrils to primary neuronal cultures, at concentrations comparative to those found in CSF (0.1 ng/ml), induced endogenous α-syn to form LB-like inclusions. This did not occur with monomeric α-syn, which is consistent with oligomeric and fibrillary α-syn species being the toxic species in PD (Volpicelli-Daley et al., Neuron 72:57-71, 2011).

A diagnosis of PD involves observation of characteristic changes in motor function including symptoms of bradykinesia, rigidity, and tremor. In PD, motor symptoms are typically preceded by non-motor or autonomic dysfunction by up to 20 years (Yu et al., Scientific Reports 8(1):567, 2018; Postuma et al., Nat. Rev. Neurol. 12(11) 622-634, 2016; Durcan et al., Eur. J. Neurol. 26(7) 979-985, 2019). One of the most prevalent non-motor features of PD is gastrointestinal (GI) dysfunction (Fasano et al., The Lancet Neurology 14(6):625-639, 2015; Noyce et al., Annals of Neurology 72(6):893-901, 2012). A number of independent longitudinal population-based studies on patients that go on to develop PD disease have shown that over 50% of PD subjects suffer GI dysfunction (Mukhtar et al., BMJ Open 8(5):e019172, 2018). Additional features of PD that precede the onset of motor symptoms include, for example, hyposmia, REM sleep behavior disorder, excessive daytime sleepiness, depression, cognitive symptoms, and autonomic nervous system dysfunction (Crosiers et al., Front. Neurol. Doi.org/10.3389/fneur.2020634490, 2021), as well as olfactory loss, decreased color vision, slowing on quantitative motor testing, and abnormal substantia nigra neuromaging findings.

There is a need for approaches to treat early, pre-motor symptoms of synucleinopathies such as PD, with the goal of alleviating the pre-motor symptoms, as well as delaying, lessening, or preventing the onset of motor symptoms.

### SUMMARY OF THE INVENTION

In one aspect, the invention provides methods of preventing, reducing, inhibiting, or slowing the development of one or more motor symptom of a synucleinopathy in a subject in need thereof, the methods including administering to the subject an effective amount of an immunotherapy targeting alpha-synuclein (α-syn).

In some embodiments, the one or more motor symptom of a synucleinopathy is selected from the group consisting of muscle rigidity, bradykinesia, tremor at rest, and postural instability.

In another aspect, the invention provides methods of treating, preventing, reducing, or inhibiting one or more gastrointestinal symptom of a synucleinopathy in a subject in need thereof, the method including administering to the subject an effective amount of an immunotherapy targeting α-syn.

In some embodiments, the one or more gastrointestinal symptom is selected from the group consisting of: drooling, salivation, dysphagia, nausea, vomiting, dyspepsia, constipation, abdominal pain, gastroparesis, and fecal incontinence.

In some embodiments, the gastrointestinal symptom occurs in the colon of the subject.

In another aspect, the invention provides methods of reducing the level of α-syn in the gastrointestinal tract (e.g., the colon) of a subject in need thereof, the method including administering to the subject an effective amount of an immunotherapy targeting α-syn.

In some embodiments, the subject does not have one or more motor symptom of a synucleinopathy or exhibits only a minimal motor symptom of a synucleinopathy.

In some embodiments, the subject does not have one or more motor symptom of a synucleinopathy selected from the group consisting of muscle rigidity, bradykinesia, tremor at rest, and postural instability.

In some embodiments, the subject has a synucleinopathy at an early, prodromal stage.

In another aspect, the invention provides methods of inducing an immune response to α-syn in a subject, inhibiting α-syn aggregation in a subject, or reducing the amount of α-syn aggregates in a subject, the method including administering an effective amount of an immunotherapy targeting α-syn to the subject, wherein the subject has a synucleinopathy at an early, prodromal stage.

In some embodiments, the synucleinopathy is selected from the group consisting of Parkinson's disease (PD), Parkinson's disease dementia (PDD), dementia with Lewy bodies (DLB), multiple system atrophy (MSA), neuroaxonal dystrophies, and pure autonomic failure (PAF).

In some embodiments, the immunotherapy comprises a peptide, a protein (e.g., an antibody), a fragment or fusion of a peptide or a protein (e.g., an antibody), or a nucleic acid molecule (e.g., an mRNA or a nucleic acid in a vector) encoding one of the molecules.

In some embodiments, the immunotherapy comprises a peptide immunogen construct.

In some embodiments, the peptide immunogen construct comprises a B cell epitope, a heterologous T cell epitope, and an optional linker.

In some embodiments, the B cell epitope induces an immune response against α-syn, e.g., when present within the peptide immunogen construct.

In some embodiments, the B cell epitope comprises a peptide of the C-terminal region of an α-syn protein, wherein the peptide optionally is about 10 to about 25 amino acids in length.

In some embodiments, the α-syn protein comprises the sequence of SEQ ID NO: 1.

In some embodiments, the B cell epitope comprises a peptide selected from a sequence of Table 1 (e.g., a peptide of any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, or 69).

In some embodiments, the heterologous T cell epitope is derived from a pathogenic protein.

In some embodiments, the heterologous T cell epitope comprises a sequence selected from a sequence of Table 2 (e.g., a sequence of any one of SEQ ID NOs: 70, 71, 72, 73, 74, 75, 76, 77, 78, 79, 80, 81, 82, 83, 84, 85, 86, 87, 88, 89, 90, 91, 92, 93, 94, 95, 96, 97, and 98).

In some embodiments, the peptide comprises a heterologous spacer or linker between the B cell epitope and the T cell epitope.

In some embodiments, the heterologous spacer or linker is selected from the group consisting of Lys-, Gly-, Lys-Lys-Lys-, (α, ε-N)Lys, and ε-N-Lys-Lys-Lys-Lys.

In some embodiments, the B cell epitope is located N-terminal to the T cell epitope.

In some embodiments, the T cell epitope is located N-terminal to the B cell epitope.

In some embodiments, the peptide immunogen construct is selected from a sequence of Table 3 (e.g., a construct of any one of SEQ ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147).

In some embodiments, the peptide immunogen construct comprises: (a) a B cell epitope comprising about 10 to about 25 amino acid residues from a C-terminal fragment of α-Syn corresponding to about amino acid G111 to about amino acid D135 of SEQ ID NO: 1; (b) a T helper epitope comprising an amino acid sequence selected from the group consisting of SEQ ID NOs: 70-98; and (c) an optional heterologous spacer selected from the group consisting of an amino acid, Lys-, Gly-, Lys-Lys-Lys-, (α, ε-N)Lys, and ε-N-Lys-Lys-Lys-Lys (SEQ ID NO: 148), wherein the B cell epitope is covalently linked to the T helper epitope directly or through the optional heterologous spacer.

In some embodiments, the B cell epitope is selected from the group consisting of SEQ ID NOs: 12-15, 17, and 49-63.

In some embodiments, the T helper epitope is selected from the group consisting of SEQ ID NOs: 70, 71, 72, 73, 74, 75, 76, 77, 78, 79, 80, 81, 82, 83, 84, 85, 86, 87, 88, 89, 90, 91, 92, 93, 94, 95, 96, 97, and 98, e.g., selected from the group consisting of SEQ ID NOs: 81, 83, and 84.

In some embodiments, the optional heterologous spacer is (α, ε-N)Lys or ε-N-Lys-Lys-Lys-Lys (SEQ ID NO: 148).

In some embodiments, the T helper epitope is covalently linked to the amino terminus of the B cell epitope.

In some embodiments, the T helper epitope is covalently linked to the amino terminus of the B cell epitope through the optional heterologous spacer.

In some embodiments, the peptide immunogen construct comprises the following formula: (Th)ₘ-(A)ₙ-(α-Syn C-terminal fragment)-X or (α-Syn C-terminal fragment)-(A)ₙ-(Th)ₘ-X wherein: Th is the T helper epitope; A is the heterologous spacer; (α-Syn C-terminal fragment) is the B cell epitope; X is an α-COOH or α-CONH2 of an amino acid; m is from 1 to about 4; and n is from 1 to about 10.

In some embodiments, the peptide immunogen construct comprises an amino acid sequence selected from the group consisting of SEQ ID NOs: 107, 108, 111-113, and 115-147.

In some embodiments, the peptide immunogen construct is in a stabilized immunostimulatory complex with a CpG oligodeoxynucleotide (ODN).

In some embodiments, the immunotherapy is comprised within a composition, which optionally comprises more than one immunotherapy, e.g., more than one peptide immunogen construct.

In some embodiments, the composition comprises peptide immunogen constructs comprising amino acid sequences of SEQ ID NOs: 112 and 113.

In some embodiments, the composition is a pharmaceutical composition comprising the immunotherapy(ies) and a pharmaceutically acceptable delivery vehicle and/or adjuvant.

In some embodiments, the composition comprises an adjuvant that comprises a mineral salt of aluminum, which optionally is selected from group consisting of Al(OH)₃ and AlPO₄.

In some embodiments, (a) the peptide immunogen construct is selected from the group consisting of SEQ ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147, e.g., selected from the group consisting of SEQ ID NOs: 107, 108, 111-113, and 115-147; and (b) the composition comprises an adjuvant that is a mineral salt of aluminum selected from the group consisting of Al(OH)₃ and AlPO₄.

In some embodiments, (a) the peptide immunogen construct is selected from the group consisting of SEQ ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147, e.g., selected from the group consisting of SEQ ID NOs: 107, 108, 111-113, and 115-147; and (b) the peptide immunogen construct is in the form of a stabilized immunostimulatory complex with a CpG ODN.

In some embodiments, the immunotherapy comprises an antibody or an epitope-binding fragment thereof that specifically binds to the B cell epitope of a peptide immunogen construct described herein (see, e.g., SEQ ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147), a B cell epitope of SEQ ID NO: 1 (e.g., the C-terminal region of SEQ ID NO: 1), or a peptide of Table 1 (e.g., any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69).

In some embodiments, the methods comprise the use of two or more, three or more, four or more, or five or more immunotherapies.

In some embodiments, the subject is diagnosed with rapid eye movement (REM) sleep behavior disorder (RBD).

In some embodiments, the subject has one or more of hyposmia, REM sleep behavior disorder, excessive daytime sleepiness, depression, cognitive symptoms, autonomic nervous system dysfunction, olfactory loss, decreased color vision, slowing on quantitative motor testing, abnormal substantia nigra neuroimaging findings, or other prodromal symptom, e.g., as described herein.

In some embodiments, the subject does not have any or any significant bradykinesia, rigidity, and/or tremor, or other symptom of a synucleinopathy that is not prodromal.

In some embodiments, the immunotherapy comprises or consists of a peptide immunogen construct that comprises or consists of SEQ ID NO: 112.

In another aspect, the invention provides compositions or kits for use in carrying out any one of the methods described herein.

In other aspects, the invention provides a composition described herein for use in treating, inhibiting, preventing, ameliorating, reducing, inhibiting, or slowing the development of one or more diseases or conditions described herein, or one or more symptoms thereof.

In other aspects, the invention provides using one or more of the compositions described herein for producing a medication for treating, inhibiting, lessening, preventing, ameliorating, reducing, inhibiting, or slowing the development of one or more diseases or conditions described herein, or one or more symptoms thereof.

In other aspects, the invention provides compositions or kits described herein for use in the preparation of medicaments for carrying out any of the methods described herein.

In other aspects, the invention provides methods for producing the compositions and kits described herein for use in treating, inhibiting, lessening, preventing, ameliorating, reducing, inhibiting, or slowing the development of one or more diseases or conditions described herein, comprising, for example, admixing the components thereof.

### BRIEF DESCRIPTION OF THE DRAWINGS

FIG. 1 Schematic representation of immunisation regime. 10-week-old Thy1SNCA/15 mice and Wt controls were administered intramuscular injections of either UB312 or adjuvant 3 weeks apart. Blood samples were collected for antibody titre analysis prior to each injection, on week 10 and week 15 (terminal time point). Behaviour tests were performed prior to the initiation of immunotherapy and at the end of the 15-week study period.

FIG. 2 Antibody titre analysis. 10-week-old Thy1SNCA/15 mice and wild type littermates were administered three intramuscular injections of either UB312 or adjuvant 3 weeks apart. Blood samples were collected prior to each injection, on week 9 and week 15 (terminal time point) and antibody titres were measured for each of the collected sera. Data points represent mean±95% Cl.

FIG. 3 Motor behaviour analysis. 10-week-old Thy1SNCA/15 mice and wild type littermates were subject to three different motor performance tests before immunisation (Pre-immunisation) and 15 weeks after the first injection (Post-immunisation). These included the challenging beam traversal test, pole test and wire hanging test. Bars represent mean±95% Cl.

FIG. 4 Immunohistochemistry for α-syn. α-Syn immunoreactivity was quantified in the cortex, hippocampus, striatum, and substantia nigra of Thy1SNCA/15 mice that received UB312 (n=12) or adjuvant (n=11). Two-tailed T test showed no difference in the mean percentage area of α-syn immunoreactivity between treatment groups. DAPI was used for visualization of cell nuclei. Scale bar=50 μm.

FIG. 5 Western blot analysis of oligomeric α-syn. α-Syn assemblies in brain homogenates from the cortex, striatum, and hippocampus of Thy1SNCA/15 mice that had received UB312 (n=14) or adjuvant (n=16) were separated by native western blot. Quantification of oligomeric and monomeric immunoreactive bands showed a significant decrease in α-syn oligomers with UB312 treatment but not monomers. Bars represent mean±95% Cl.

FIG. 6 Immunohistochemistry for microglia. Iba1 immunoreactivity was quantified in the cortex, hippocampus, striatum and substantia nigra of Thy1SNCA/15 mice that received UB312 (n=11) or adjuvant (n=9) or Wt littermates that received adjuvant (n=8). One-way ANOVA with Bonferroni corrections showed a significant increase in the mean percentage area of Iba1 in the substantia nigra. Bars represent mean±95% Cl.

FIG. 7 Immunohistochemistry for astrocytes. GFAP immunoreactivity was quantified in the cortex, hippocampus, striatum, and substantia nigra of Thy1SNCA/15 mice that received UB312 (n=11) or adjuvant (n=9) or Wt littermates that received adjuvant (n=8). One-way ANOVA showed no difference between treatment groups in each brain region. Bars represent mean±95% Cl.

FIG. 8 Immunohistochemistry for endothelial activation and T cell infiltration. ICAM1 immunoreactivity was quantified in the cortex, hippocampus, striatum, and substantia nigra of Thy1SNCA/15 mice that received UB312 (n=11) or adjuvant (n=9) or Wt littermates that received adjuvant (n=8). One-way ANOVA showed no difference between treatment groups in each brain region. CD3+ T cells were counted in whole brain sections. Bars represent mean±95% Cl.

FIG. 9 Immunohistochemistry for α-syn and enteric glial cell (EGC) activation in the gastrointestinal tract. Immunofluorescence shows α-syn immunoreactivity in the muscularis (arrows) of the duodenum and colon with DAPI counterstain. Two-tailed t-test showed a significant reduction in the mean percentage area of α-syn in the colon muscularis after UB312 immunotherapy compared to adjuvant in Thy1SNCA/15 mice. EGC reactivity was measured by quantifying GFAP immunoreactivity in myenteric ganglia. One-way ANOVA showed a significant reduction in the mean percentage area of GFAP in the colon of Thy1SNCA/15 mice receiving UB312 when compared to adjuvant or Wt littermates receiving adjuvant. There was no effect of UB312 immunotherapy in the duodenum on either α-syn or GFAP expression levels. Bars represent mean±95% Cl.

FIGS. 10A and 10B Optimisation of antibody concentration for western blot analysis. FIG. 10A, Determining the linear range of α-syn (MJFR1) and Total protein (Revert, Licor). Brain homogenates were loaded in ascending concentration from 1-50 μg of protein in a 12% native gel. Quantification of α-syn immunoreactive bands and total protein is shown on the right. FIG. 10B, transmission electron micrograph (TEM) of in house synthesised α-syn monomers (Right) compared to after they were allowed to form fibrils (left). The monomeric α-syn was used as a molecular weight marker for western blot analysis.

### DETAILED DESCRIPTION OF THE INVENTION

The present disclosure is based, in part, on the discovery that immunotherapy directed against alpha-synuclein (α-syn) can be used to prevent or reduce the onset or early development of motor symptoms characteristic of synucleinopathies, as well as to decrease α-syn levels in the gastrointestinal tract.

Accordingly, the disclosure is directed to methods of preventing or reducing the development of one or more motor symptoms of a synucleinopathy in a subject in need thereof by using immunotherapy to target α-syn. The present disclosure is also directed to methods of treating, preventing, reducing, or inhibiting one or more gastrointestinal symptom or asymptomatic gastrointestinal pathology of a synucleinopathy using such approaches. Further, the disclosure is directed to methods of reducing the levels of α-syn in the gastrointestinal tract of a subject in need thereof using such approaches. Advantageously, in some embodiments, the methods of the disclosure can be used to treat subjects in whom the development of a synucleinopathy is at an early stage, which can provide substantial benefits for inhibiting or slowing the development of the disease, leading to improved quality of life and prolonged health span.

The methods of the disclosure, including molecules and compositions used therein, are described in an exemplary manner below.

The section headings used herein are for organizational purposes only and are not to be construed as limiting the subject matter described. All references or portions of references cited in this application are expressly incorporated herein by reference in their entirety for any purpose.

Unless otherwise indicated, all technical and scientific terms used herein have the same meaning as commonly understood by those of ordinary skill in the art to which this invention belongs. The singular terms “a,” “an,” and “the” include the respective plural terms, unless context indicates otherwise. Similarly, the word “or” is intended to include “and” unless the context clearly indicates otherwise. Hence “comprising A or B” means including A or B, or A and B. It is further to be understood that all amino acid sizes, and all molecular weight or molecular mass values given for polypeptides are approximate. Although methods and materials similar or equivalent to those described herein can be used in the practice or testing of the disclosed methods, suitable methods and materials are described below.

All publications, patent applications, patents, and other references mentioned herein are incorporated by reference herein in their entirety. In case of conflict, the present specification, including explanations of terms, will control. In addition, the materials, methods, and examples are illustrative only and not intended to be limiting.

### Immunotherapeutic Agents and Compositions

The methods of the disclosure can include the use of, for example, one or more peptide, protein (e.g., an antibody), a fragment or fusion of a peptide or a protein (e.g., an antibody), or a nucleic acid molecule (e.g., mRNA or nucleic acid in a viral vector) encoding one of said molecules, wherein the molecule is directed against α-syn.

### Peptides

In some embodiments, the methods of the disclosure employ a peptide immunogen construct including a B cell epitope from α-syn linked to a heterologous T helper cell (Th) epitope, directly or through an optional heterologous spacer. Constructs such as these are described, e.g., in WO 2018/232369, the contents of which are incorporated herein by reference.

The B cell epitope portion of the peptide immunogen constructs can optionally include about 10 to about 25 amino acid residues from the C-terminal end of α-syn, corresponding, e.g., to the sequence from about the glycine at amino acid position 111 (G111) to about the asparagine at amino acid position 135 (D135) of full-length α-syn (SEQ ID NO: 1). The heterologous Th epitope portion of the peptide immunogen constructs can optionally be derived from pathogenic proteins. The B cell epitope and Th epitope portions of the peptide immunogen constructs act together when administered to a subject to stimulate the generation of antibodies that specifically recognize and bind to the α-syn B cell epitope portion of the constructs.

Accordingly, the phrase “α-syn peptide immunogen construct,” as used herein, refers to a peptide containing (a) a B cell epitope having about 10 to about 25 amino acid residues from the C-terminal end of α-syn, corresponding to the sequence from about the glycine at amino acid position 111 (G111) to about the asparagine at amino acid position 135 (D135) of full-length α-syn (SEQ ID NO: 1); (b) a heterologous Th epitope; and (c) an optional heterologous spacer.

In certain embodiments, the peptide immunogen construct can be represented by the formulae: (Th)ₘ-(A)ₙ-(α-Syn C-terminal fragment)-X or (α-Syn C-terminal fragment)-(A)ₙ-(Th)ₘ-X, wherein Th is a heterologous T helper epitope; A is a heterologous spacer; (α-Syn C-terminal fragment) is a B cell epitope having about 10 to about 25 amino acid residues from the C-terminal end of α-Syn; X is an α-COOH or α-CONH₂ of an amino acid; m is an integer from 1 to about 4; and n is an integer from 0 to about 10. In some embodiments, A is an amino acid and n indicates the number of amino acids, wherein each A can be identical to one another or one or more of the A's can be different amino acids. The various components of the disclosed α-syn peptide immunogen construct are described below.

### α-Syn and α-Syn C-Terminal Fragments

The terms “α-syn,” “alpha-synuclein,” “α-synuclein,” and the like, as used herein, refer to (a) the full-length α-syn protein and/or (b) fragments thereof from any organism that expresses α-syn. In some embodiments, the α-syn protein is human. In certain embodiments, the full-length human α-syn protein has 140 amino acids (Accession No. NP_000336) (SEQ ID NO: 1).

The phrase “C-terminal region” or “C-terminal end” of α-syn, as used herein, refers to any amino acid sequence from the carboxyl-terminal portion of α-syn. In certain embodiments, the C-terminal region or C-terminal end of α-syn relates to the amino acid sequence between residues 96-140, or fragments thereof, of α-syn.

The phrase “α-syn C-terminal fragment” or “B cell epitope from the C-terminal end of α-syn,” as used herein, refers to a portion of the full-length α-syn sequence that includes about 10 to about 25 amino acid residues from the C-terminal end of α-syn, corresponding to the sequence from about the glycine at amino acid position 111 (G111) to about the asparagine at amino acid position 135 (D135) of full-length α-syn. The α-syn C-terminal fragment is also referred to herein as the α-syn G111-D135 peptide and fragments thereof. The various α-syn C-terminal fragments described herein are referred to by their amino acid positions in relation to the full-length sequence of α-syn represented by SEQ ID NO: 1.

In some embodiments, the α-syn C-terminal fragment is the 25 amino acid α-syn G111-D135 peptide represented by SEQ ID NO: 12. In other embodiments, the α-syn C-terminal fragment contains about 10 contiguous amino acids of the α-syn G111-D135 peptide represented by SEQ ID NO: 12. In certain embodiments, the α-syn C-terminal fragment includes 10, 11, 12, 13, 14, 15, 16, 17, 18, 19, 20, 21, 22, 23, 24, or 25 contiguous amino acids of the α-syn G111-D135 peptide represented by SEQ ID NO: 12. In some embodiments, the α-syn C-terminal fragment has a sequence selected from any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69. In specific embodiments, the α-syn C-terminal fragment has an amino acid sequence represented by one of SEQ ID NOs: 12-15, 17, or 49-64, as shown in Table 1.

In some embodiments, the B cell epitope of the peptide immunogen construct comprises or consists of a peptide of any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, or 69.

The α-syn C-terminal fragment of the present disclosure also includes immunologically functional analogues or homologues of the α-syn G111-D135 peptide, or fragments thereof. Functional immunological analogues or homologues of α-syn G111-D135 peptide or fragments thereof include variants that retain substantially the same immunogenicity as the original peptide. Immunologically functional analogues can have one or more conservative substitutions in an amino acid position; a change in overall charge; a covalent attachment to another moiety; or amino acid additions, insertions, or deletions; and/or any combination thereof.

Conservative substitutions are those in which one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

Immunologically functional analogues include amino acid sequences that comprise conservative substitutions, additions, deletions, or insertions from one to about four amino acid residues that elicit immune responses that are cross-reactive with the α-syn G111-D135 peptide. The conservative substitutions, additions, and insertions can be accomplished with natural or non-natural amino acids. Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-aminohexanoic acid), hydroxyproline, mercaptopropionic acid (MPA), 3-nitro-tyrosine, pyroglutamic acid, and the like. Naturally occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

In one embodiment, the immunologically functional analogue of a particular peptide includes the same amino acid sequence as the original peptide and further includes three lysine residues (Lys-Lys-Lys) added to the amino terminus of the α-syn G111-D135 peptide (or a fragment thereof) B cell epitope peptide. In this embodiment, the addition of three lysine residues to the original peptide sequence changes the overall charge of the original peptide but does not alter the function of the original peptide.

Functional analogs or homologues of the other peptides described herein (see list above and Table 1; e.g., any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69) are also included.

In certain embodiments, a functional analogue of the α-syn C-terminal fragment has at least 50% sequence identity to the original amino acid sequence. In other embodiments, the functional analogue has at least 80% identity to the original amino acid sequence. In yet other embodiments, the functional analogue has at least 85% identity to the original amino acid sequence. In still other embodiments, the functional analogue has at least 90% or at least 95% identity to the original amino acid sequence. The percent identity between two sequences can be determined manually by inspection of the two optimally aligned sequences or by using software programs or algorithms (e.g., BLAST, ALIGN, CLUSTAL) using standard parameters, as is known in the art.

### Heterologous T Helper Cell Epitopes (Th Epitopes)

Peptide immunogen constructs used in the methods of disclosure include a B cell epitope from α-syn covalently linked to a heterologous T helper cell (Th) epitope directly or through an optional heterologous spacer. The heterologous Th epitope in the α-syn peptide immunogen construct enhances the immunogenicity of the α-syn C-terminal fragment, which facilitates the production of specific high titer antibodies directed against the optimized target B cell epitope (i.e., the α-syn C-terminal fragment) through rational design.

The term “heterologous,” as used herein, refers to an amino acid sequence that is derived from an amino acid sequence that is not part of, or homologous with, the wild-type sequence of α-syn. Thus, a heterologous Th epitope is a Th epitope derived from an amino acid sequence that is not naturally found in α-syn (i.e., the Th epitope is not autologous to α-syn). Since the Th epitope is heterologous to α-syn, the natural amino acid sequence of α-syn is not extended in either the N-terminal or C-terminal directions when the heterologous Th epitope is covalently linked to the α-syn C-terminal fragment.

The heterologous Th epitope of the present disclosure can be any Th epitope that does not have an amino acid sequence naturally found in α-syn. The Th epitope can have an amino acid sequence derived from any species (e.g., human, pig, cattle, dog, rat, mouse, guinea pigs, etc.) or from a pathogen (e.g., a measles virus or a hepatis virus (e.g., a hepatitis virus surface protein; see below). The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e., little, if any, of the antibodies generated by the α-syn peptide immunogen constructs will be directed towards the Th epitope, thus allowing a very focused immune response directed to the targeted B cell epitope of the α-syn C-terminal fragment.

Th epitopes include, but are not limited to, amino acid sequences derived from foreign pathogens, as exemplified in Table 2 (SEQ ID NOs: 70-98). Further, Th epitopes include idealized artificial Th epitopes and combinatorial idealized artificial Th epitopes (e.g., SEQ ID NOs: 71 and 78-84). The heterologous Th epitope peptides presented as a combinatorial sequence (e.g., SEQ ID NOs: 79-82), contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 79-82, which are shown in Table 2. Th epitope peptides of the present invention provide broad reactivity and immunogenicity to animals and patients from genetically diverse populations.

The Th epitopes of the peptide immunogen constructs can therefore be selected from any one of SEQ ID NOs: 70, 71, 72, 73, 74, 75, 76, 77, 78, 79, 80, 81, 82, 83, 84, 85, 86, 87, 88, 89, 90, 91, 92, 93, 94, 95, 96, 97, and 98, and also immunologically functional analogues thereof (see below).

α-Syn peptide immunogen constructs comprising Th epitopes can be produced simultaneously in a single solid-phase peptide synthesis in tandem with the α-syn C-terminal fragment. Th epitopes also include immunological analogues of Th epitopes. Immunological Th analogues include immune-enhancing analogs, cross-reactive analogues and fragments of any of these Th epitopes that are sufficient to enhance or stimulate an immune response to the α-syn C-terminal fragments.

Immunologically functional analogues of the Th epitope peptides are also effective and can be used in the methods of the disclosure. Immunologically functional Th analogues can include conservative substitutions, additions, deletions, and insertions of from one to about five amino acid residues in the Th epitope which do not essentially modify the Th-stimulating function of the Th epitope. The conservative substitutions, additions, and insertions can be accomplished with natural or non-natural amino acids, as described above for the α-syn C-terminal fragments. Table 2 identifies another variation of a functional analogue for Th epitope peptide. In particular, SEQ ID NOs: 71 and 78 of MvF1 and MvF2 Th are functional analogues of SEQ ID NOs: 81 and 83 of MvF4 and MvF5 in that they differ in the amino acid frame by the deletion (SEQ ID NOs: 71 and 78) or the inclusion (SEQ ID NOs: 81 and 83) of two amino acids each at the N- and C-termini. The differences between these two series of analogous sequences would not affect the function of the Th epitopes contained within these sequences. Therefore, functional immunological Th analogues can, for example, include several versions of the Th epitope derived from Measles Virus Fusion protein MvF1-4 Ths (SEQ ID NOs: 71, 78, 79, 81, and 83) and from Hepatitis Surface protein HBsAg 1-3 Ths (SEQ ID NOs: 80, 82, and 84).

The Th epitope in the α-syn peptide immunogen construct can be covalently linked at either N- or C-terminal end of the α-syn C-terminal peptide. In some embodiments, the Th epitope is covalently linked to the N-terminal end of the α-syn C-terminal peptide. In other embodiments, the Th epitope is covalently linked to the C-terminal end of the α-syn C-terminal peptide. In certain embodiments, more than one Th epitope is covalently linked to the α-syn C-terminal fragment. When more than one Th epitope is linked to the α-syn C-terminal fragment, each Th epitope can have the same amino acid sequence or different amino acid sequences. In addition, when more than one Th epitope is linked to the α-syn C-terminal fragment, the Th epitopes can be arranged in any order. For example, the Th epitopes can be consecutively linked to the N-terminal end of the α-syn C-terminal fragment, or consecutively linked to the C-terminal end of the α-syn C-terminal fragment, or a Th epitope can be covalently linked to the N-terminal end of the α-syn C-terminal fragment while a separate Th epitope is covalently linked to the C-terminal end of the α-syn C-terminal fragment. There is no limitation in the arrangement of the Th epitopes in relation to the α-syn C-terminal fragment.

In some embodiments, the Th epitope is covalently linked to the α-syn C-terminal fragment directly. In other embodiments, the Th epitope is covalently linked to the α-syn C-terminal fragment through a heterologous spacer described in further detail below.

### Heterologous Spacer

The α-syn peptide immunogen constructs optionally include a heterologous spacer that covalently links the B cell epitope from α-syn to the heterologous T helper cell (Th) epitope.

As discussed above, the term “heterologous,” refers to an amino acid sequence that is derived from an amino acid sequence that is not part of, or homologous with, the wild-type sequence of α-syn. Thus, the natural amino acid sequence of α-syn is not extended in either the N-terminal or C-terminal directions when the heterologous spacer is covalently linked to the B cell epitope from α-syn because the spacer is heterologous to the α-syn sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the α-syn peptide immunogen construct. Structurally, the spacer provides a physical separation of the Th epitope from the B cell epitope of the α-syn C-terminal fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences of flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 149), where Xaa is any amino acid, for example, aspartic acid.

The spacer can also provide functional features to the α-syn peptide immunogen construct. For example, the spacer can be designed to change the overall charge of the α-syn peptide immunogen construct, which can affect the solubility of the peptide immunogen construct. Additionally, changing the overall charge of the α-syn peptide immunogen construct can affect the ability of the peptide immunogen construct to associate with other compounds and reagents. As discussed in further detail below, the α-syn peptide immunogen construct can be formed into a stable immunostimulatory complex with a highly charged oligonucleotide, such as CpG oligomers through electrostatic association. The overall charge of the α-syn peptide immunogen construct is important for the formation of these stable immunostimulatory complexes.

Chemical compounds that can be used as a spacer include, but are not limited to (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine.

Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-aminohexanoic acid), hydroxyproline, mercaptopropionic acid (MPA), 3-nitro-tyrosine, pyroglutamic acid, and the like.

The spacer in the α-syn peptide immunogen construct can be covalently linked at either N- or C-terminal end of the Th epitope and the α-syn C-terminal peptide. In some embodiments, the spacer is covalently linked to the C-terminal end of the Th epitope and to the N-terminal end of the α-syn C-terminal peptide. In other embodiments, the spacer is covalently linked to the C-terminal end of the α-syn C-terminal peptide and to the N-terminal end of the Th epitope. In certain embodiments, more than one spacer can be used, for example, when more than one Th epitope is present in the peptide immunogen construct. When more than one spacer is used, each spacer can be the same as each other or different. Additionally, when more than one Th epitope is present in the peptide immunogen construct, the Th epitopes can be separated with a spacer, which can be the same as, or different from, the spacer used to separate the Th epitope from the B cell epitope. There is no limitation in the arrangement of the spacer in relation to the Th epitope or the α-syn C-terminal fragment.

In certain embodiments, the heterologous spacer is a naturally occurring amino acid or a non-naturally occurring amino acid. In other embodiments, the spacer contains more than one naturally occurring or non-naturally occurring amino acid (e.g., the spacer is a peptide). The spacer may comprise one or more Lys (e.g., 1, 2, 3, 4, 5, or 6) and/or one or more Gly (e.g., 1, 2, 3, 4, 5, or 6). In specific embodiments, the spacer is Lys-, Gly-, Lys-Lys-Lys-, (α, ε-N)Lys, or ε-N-Lys-Lys-Lys-Lys (SEQ ID NO: 148).

### Specific Embodiments of the α-Syn Peptide Immunogen Construct

The α-syn peptide immunogen construct can be represented by the formulae: (Th)ₘ-(A)ₙ-(α-syn C-terminal fragment)-X or (α-syn C-terminal fragment)-(A)ₙ-(Th)ₘ-X, wherein Th is a heterologous T helper epitope; A is a heterologous spacer; (α-Syn C-terminal fragment) is a B cell epitope having about 10 to about 25 amino acid residues from the C-terminal end of α-Syn; X is an α-COOH or α-CONH₂ of an amino acid; m is an integer from 1 to about 4; and n is an integer from 0 to about 10. In some embodiments, A is an amino acid and n indicates the number of amino acids, wherein each A can be identical to one another or one or more of the A's can be different amino acids.

In certain embodiments, the heterologous Th epitope in the α-syn peptide immunogen construct has an amino acid sequence selected from any of SEQ ID NOs: 70-98, or combinations thereof, shown in Table 2. In specific embodiments, the Th epitope has an amino acid sequence selected from any of SEQ ID NOs: 78-84. In certain embodiments, the α-syn peptide immunogen construct contains more than one Th epitope.

In certain embodiments, the optional heterologous spacer is selected from any of Lys-, Gly-, Lys-Lys-Lys-, (α, ε-N)Lys, ε-N-Lys-Lys-Lys-Lys (SEQ ID NO: 148), and combinations thereof. In specific embodiments, the heterologous spacer is ε-N-Lys-Lys-Lys-Lys (SEQ ID NO: 148).

In certain embodiments, the α-Syn C-terminal fragment has about 10 to about 25 amino acid residues from the C-terminal end of α-syn, corresponding to the sequence from about the glycine at amino acid position 111 (G111) to about the asparagine at amino acid position 135 (D135) of full-length α-syn. In some embodiments, the α-syn C-terminal fragment has an amino acid sequence of any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69. In specific embodiments, the α-syn C-terminal fragment has an amino acid sequence represented by SEQ ID NOs: 12-15, 17, or 49-64, as shown in Table 1.

In certain embodiments, the α-syn peptide immunogen construct has an amino acid sequence from Table 3, e.g., a sequence selected from any of SEQ ID NOs: 107-108, 111-113, and 115-147, as shown in Table 3. In specific embodiments, the α-Syn peptide immunogen construct has an amino acid sequence selected from any of SEQ ID NOs: 107-108 and 111-113.

In some embodiments, the α-syn peptide immunogen construct can comprise or consist of any one of SEQ ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147 as well as homologues, analogues, fragments, and/or combinations thereof.

### Compositions

Peptide immunogen constructs can be comprised with compositions, including pharmaceutical compositions, which are capable of eliciting an immune response and the production of antibodies against the peptide immunogen constructs in a subject (e.g., a human patient). The disclosed compositions can include one or a mixture of more than one of the peptide immunogen constructs. Furthermore, in some embodiments, the compositions include the peptide immunogen construct(s) together with one or more additional component, e.g., carriers, adjuvants, buffers, and other suitable reagents. In some embodiments, the compositions include the peptide immunogen constructs in the form of a stabilized immunostimulatory complex with a CpG oligomer that is optionally supplemented with an adjuvant.

Compositions containing a disclosed α-syn peptide immunogen construct can be in liquid or solid form. Liquid compositions can include water, buffers, solvents, salts, and/or any other acceptable reagent that does not alter the structural or functional properties of the α-syn peptide immunogen construct. Peptide compositions can contain one or more of the disclosed α-syn peptide immunogen constructs.

### Pharmaceutical Compositions

The methods of the present disclosure can utilize pharmaceutical compositions containing the disclosed α-syn peptide immunogen construct(s).

Pharmaceutical compositions can contain carriers and/or other additives in a pharmaceutically acceptable delivery system. Accordingly, pharmaceutical compositions can contain a pharmaceutically effective amount of an α-syn peptide immunogen construct together with pharmaceutically acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the α-syn peptide immunogen construct without having any specific antigenic effect itself. Adjuvants used in the pharmaceutical composition can include oils, aluminum salts, virosomes, aluminum phosphate (e.g., ADJU-PHOS®), aluminum hydroxide (e.g., ALHYDROGEL®), liposyn, saponin, squalene, L121, Emulsigen®, monophosphoryl lipid A (MPL), QS21, ISA 35, ISA 206, ISA50V, ISA51, ISA 720, as well as the other adjuvants and emulsifiers.

In some embodiments, the pharmaceutical composition contains Montanide™ ISA 51 (an oil adjuvant composition comprised of vegetable oil and mannide oleate for production of water-in-oil emulsions), Tween® 80 (also known as polysorbate 80 or polyoxyethylene (20) sorbitan monooleate), a CpG oligonucleotide, and/or any combination thereof. In other embodiments, the pharmaceutical composition is a water-in-oil-in-water (i.e., w/o/w) emulsion with Emulsigen or Emulsigen D as the adjuvant.

Pharmaceutical compositions can be formulated for immediate release or for sustained release. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the α-syn peptide immunogen construct can also be prepared prior to injection. The pharmaceutical composition can be administered by any suitable mode of application, for example, intramuscularly, subcutaneously, intradermally, intravenously, intraperitoneally, intranasally, orally, etc. and by use of any suitable formulation or delivery device.

Pharmaceutical compositions can also be formulated in a suitable dosage unit form. In some embodiments, the pharmaceutical composition contains from about 0.5 μg to about 1 mg of the α-syn peptide immunogen construct per kg body weight. In some embodiments, the pharmaceutical composition contains 10-1000 μg, e.g., 20-500 μg, 50-400 μg, or 100-300 μg of an immunotherapy as described herein (e.g., a peptide immunogen construct). Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human, but non-human mammals, including transgenic mammals can also be treated. When delivered in multiple doses, the pharmaceutical compositions may be conveniently divided into an appropriate amount per dosage unit form as determined to be appropriate by one skilled in the art. The administered dosage will depend on the age, weight, and general health of the subject as is well known in the therapeutic arts.

In some embodiments, the pharmaceutical composition contains more than one α-syn peptide immunogen construct and/or antibody. A pharmaceutical composition containing a mixture of more than one α-syn peptide immunogen construct (and/or antibody) to allow for synergistic enhancement of the immunoefficacy of the constructs. Pharmaceutical compositions containing more than one α-syn peptide immunogen construct can be more effective in a larger genetic population due to a broad MHC class II coverage thus provide an improved immune response to the α-syn peptide immunogen constructs.

In some embodiments, the pharmaceutical composition contains an α-syn peptide immunogen construct selected from SEQ ID NOs: 107, 108, 111-113, and 115-147, as well as homologues, analogues, fragments, and/or combinations thereof. In specific embodiments, pharmaceutical compositions contain an α-syn peptide immunogen construct selected from SEQ ID NOs: 107, 108, 111-113, and any combination thereof.

In some embodiments, the α-syn peptide immunogen construct can comprise or consist of any one of SEQ ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147 as well as homologues, analogues, fragments, and/or combinations thereof.

Pharmaceutical compositions containing an α-syn peptide immunogen construct can be used to elicit an immune response and to produce antibodies in a subject upon administration. In some embodiments, a pharmaceutical composition as described herein is administered to a subject 1, 2, 3, 4, 5, or more times as determined to be appropriate by those of skill in the art. The compositions can be administered, for example, in an initial dose, followed by 1 or more (e.g., 2, 3, 4, 5, or more) booster doses. In some embodiments, an initial dose is administered in week 1 and then is followed by a dose at week 5 and an additional dose at week 13. In some embodiments, the amount of each dose is the same (e.g., 100 μg or 300 μg; also see above). In some embodiments, the amount of each dose varies, as can be determined to be appropriate by those of skill in the art. For example, the initial dose may be 40 μg, followed by doses for 100 μg, 300 μg, or 1000 μg in subsequent administrations (e.g., at weeks 5 and 13).

### Immunostimulatory Complexes

The methods of the present disclosure can also utilize pharmaceutical compositions containing an α-syn peptide immunogen construct in the form of an immunostimulatory complex with a CpG oligonucleotide. Such immunostimulatory complexes are specifically adapted to act as an adjuvant and as a peptide immunogen stabilizer. The immunostimulatory complexes are in particulate form, which can efficiently present the α-syn peptide immunogen to the cells of the immune system to produce an immune response. The immunostimulatory complexes may be formulated as a suspension for parenteral administration. The immunostimulatory complexes may also be formulated in the form of water in oil emulsions, as a suspension in combination with a mineral salt or with an in-situ gelling polymer for the efficient delivery of the α-syn peptide immunogen to the cells of the immune system of a subject following parenteral administration.

The stabilized immunostimulatory complex can be formed by complexing an α-syn peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the α-syn peptide immunogen construct is designed to contain a cationic portion that is positively charged at a pH in the range of 5.0 to 8.0. The net charge on the cationic portion of the α-syn peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the α-syn peptide immunogen construct and expressed as the net average charge. A suitable peptide immunogen has a cationic portion with a net average positive charge of +1. In some embodiments, the peptide immunogen has a net positive charge in the range that is larger than +2. In some embodiments, the cationic portion of the α-syn peptide immunogen construct is the heterologous spacer. In certain embodiments, the cationic portion of the α-syn peptide immunogen construct has a charge of +4 when the spacer sequence is (α, ε-N)Lys, ε-N-Lys-Lys-Lys-Lys (SEQ ID NO: 148).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. In some embodiments, the CpG immunostimulatory single-stranded DNA molecules contain 18-48 nucleotide bases, with the number of repeats of CpG motif in the range of 3 to 8.

In some embodiments, the anionic oligonucleotide is represented by the formula: 5′X¹CGX²3′ wherein C and G are unmethylated; and X¹ is selected from the group consisting of A (adenine), G (guanine) and T (thymine); and X² is C (cytosine) or T (thymine). In other embodiments, the anionic oligonucleotide is represented by the formula: 5′ (X³)2CG(X⁴)23′ wherein C and G are unmethylated; and X³ is selected from the group consisting of A, T or G; and X⁴ is C or T.

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

The α-syn peptide immunogen constructs used in the methods of the disclosure can be made using chemical synthesis methods that are well known in the art (see, e.g., Fields et al., Chapter 3 in Synthetic Peptides: A User's Guide, ed. Grant, W. H. Freeman & Co., New York, NY, 1992, p. 77). For example, the α-syn peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of α-syn peptide immunogen constructs comprising combinatorial library peptides for Th epitopes can be accomplished by providing a mixture of alternative amino acids for coupling at a given variable position. After complete assembly of a desired α-syn peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to those of skill in the art.

The quality of peptides produced by this chemical process can be controlled and defined and, as a result, reproducibility of α-syn peptide immunogen constructs, immunogenicity, and yield can be assured. Detailed description of the manufacturing of an α-syn peptide immunogen construct through solid phase peptide synthesis is described in Example 1 of WO 2018/232369.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically derived drugs. Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The α-syn peptide immunogen constructs can also be made using recombinant DNA technology including by the use of nucleic acid molecules, vectors, and/or host cells. As such, nucleic acid molecules encoding the α-syn peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the α-syn peptide immunogen construct and immunologically functional analogues of the α-syn G111-D135 fragment derived peptide immunogen constructs. For example, methods can include a step of incubating a host cell containing an expression vector containing a nucleic acid molecule encoding an α-syn peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

### Methods for Manufacturing of Immunostimulatory Complexes

As noted above, the methods of the disclosure can further employ immunostimulatory complexes comprising α-syn peptide immunogen constructs and CpG oligodeoxynucleotide (ODN) molecules. Stabilized immunostimulatory complexes (ISC) are derived from a cationic portion of the α-syn peptide immunogen construct and a polyanionic CpG ODN molecule. The self-assembling system is driven by electrostatic neutralization of charge. Stoichiometry of the molar charge ratio of cationic portion of the α-syn peptide immunogen construct to anionic oligomer determines extent of association. The non-covalent electrostatic association of α-syn peptide immunogen construct and CpG ODN is a completely reproducible process. The peptide/CpG ODN immunostimulatory complex aggregates, which facilitate presentation to the “professional” antigen-presenting cells (APC) of the immune system thus further enhancing of the immunogenicity of the complexes. These complexes are easily characterized for quality control during manufacturing. The peptide/CpG ISC are well tolerated in vivo. This particulate system comprising CpG ODN and α-syn G111-D135 fragment derived peptide immunogen constructs was designed to take advantage of the generalized B cell mitogenicity associated with CpG ODN use and yet promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen-presenting cells (APC). Consequently, such formulations are novel conceptually and offer potential advantages by promoting the stimulation of immune responses by alternative mechanisms.

### Antibodies

The methods of the disclosure can utilize antibodies that specifically recognize and bind to α-syn, for example, to a C-terminal peptide of α-syn, e.g., a B cell epitope portion of the peptide immunogen constructs described herein (also see WO 2018/232369). Antibodies for use in therapy can be generated using standard methods in the art and include, e.g., monoclonal antibodies, polyclonal antibodies, multispecific antibodies (e.g., bispecific and trispecific antibodies), and antibody fragments, provided that the desired antigen-binding activity and specificity is maintained. Antibody fragments include, for example, Fv, single-chain Fv (scFv), Fab, Fab′, di-scFv, sdAb (single domain antibody), and (Fab′)₂ (including a chemically linked F(ab′)₂). Antibodies also include, e.g., chimeric antibodies, humanized antibodies, and antibodies of various species such as mouse, human, cynomolgus monkey, etc. Furthermore, antibody variants having the sequences from other organisms are also included. Antibody fragments also include either orientation of single chain scFvs, tandem di-scFv, diabodies, tandem tri-sdcFv, minibodies, etc. Antibody fragments further include nanobodies (sdAb, an antibody having a single, monomeric domain, such as a pair of variable domains of heavy chains, without a light chain). An antibody fragment can be referred to as being a specific species in some embodiments (for example, human scFv or a mouse scFv). This denotes the sequences of at least part of the non-CDR regions, rather than the source of the construct.

### Methods of Treatment

The present disclosure provides methods for treating, delaying, lessening, and/or preventing synucleinopathies using the disclosed immunotherapies (e.g., peptide immunogen constructs and/or antibodies directed against the peptide immunogen constructs). In some embodiments, the methods include administering to a subject a composition containing a disclosed peptide immunogen construct and/or antibody. In certain embodiments, the compositions utilized in the methods contain a disclosed peptide immunogen construct in the form of a stable immunostimulatory complex with negatively charged oligonucleotides, such as CpG oligomers, through electrostatic association, which complexes are further supplemented, optionally, with mineral salts or oil as adjuvant, for administration to subjects with synucleinopathies. The disclosed methods also include dosing regimens, dosage forms, and routes for administering the peptide immunogen constructs to a subject at risk for, or with, a synucleinopathy.

Subjects who can be treated according to the methods of the disclosure include patients, such as human patients, who have or are at risk of developing a synucleinopathy such as, for example, Parkinson's disease (PD), Parkinson's disease dementia (PDD), dementia with Lewy bodies (DLB), multiple system atrophy (MSA), neuroaxonal dystrophies, pure autonomic failure (PAF).

In some embodiments, subjects treated according to methods of the disclosure are at an early stage in the development of a synucleinopathy. For example, the subjects may be in what is known in the art as a “prodromal” stage, in which early signs and symptoms of the disease may appear, but cardinal symptoms of the disease (e.g., motor symptoms) are not yet present. Identification of subjects at the prodromal stage typically involves consideration of combinations of clinical, fluid, tissue, genetic, and imaging features or markers. For example, imaging by, e.g., positron emission tomography (PET), single photon emission computed tomography (SPECT), or magnetic resonance imaging (MRI) can be used. In some embodiments, PET or SPECT is used for detecting dopamine transporter (DAT) (DAT-PET or DAT-SPECT). An additional approach that can be used is detection of pathological α-syn in cerebrospinal fluid or tissue biopsies by protein misfolding cyclic amplification (PMCA). In other examples, skin tests can be used for detection of, e.g., phosphorylated α-syn (e.g., using the Syn-One Test™) or sebum lipids (Sinclair et al., Nature 12:1592, 2021). In addition to these tests, a subject can be identified by detection of prodromal symptoms of the synucleinopathy including, e.g., REM-sleep behavior disorder, hyposmia, constipation, mood disorders, excessive daytime somnolence, global cognitive deficit, small handwriting, restless leg syndrome, orthostatic hypotension, sexual dysfunction, urinary dysfunction, voice and face akinesia, or combinations thereof. Furthermore, family history can be a useful consideration. Additionally, a score for subthreshold parkinsonism, such as a UPDRS score for prodromal PD (Goetz et al., Mov. Disord. 27:1239-1242, 2012), can be determined. Markers such as α-syn, neurofilament light chain (NfL), and plasma urate levels can also be assessed. Genetic markers (e.g., mutations in LRRK2, GBA, SNCA, and/or VPS35 genes) can additionally be used. Subjects who are in an early (e.g., prodromal) stage of a synucleinopathy can be treated according to the methods of the disclosure. In some embodiments, the subject is diagnosed with REM-sleep behavior disorder but does not have any or any significant motor symptom (e.g., bradykinesia, rigidity, and/or tremor). In some embodiments, the subject has one or more of hyposmia, REM sleep behavior disorder, excessive daytime sleepiness, depression, cognitive symptoms, autonomic nervous system dysfunction, olfactory loss, decreased color vision, slowing on quantitative motor testing, and abnormal substantia nigra neuroimaging findings. In some embodiments, the subject does not have any or any significant motor symptom (e.g., bradykinesia, rigidity, and/or tremor).

The present disclosure also includes methods of using pharmaceutical compositions containing α-syn peptide immunogen constructs. In certain embodiments, the pharmaceutical compositions containing α-syn peptide immunogen constructs can be used for: (a) inhibiting α-syn aggregation in a subject; (b) inducing disaggregate of preformed α-syn aggregates in a subject; (c) reducing microglial TNF-alpha and IL6 secretion in a subject; (d) reducing neurodegeneration triggered by exogeneous α-syn aggregates in a subject; (e) reducing neurodegeneration in α-syn overexpressing cells; (f) reducing serum α-syn levels in a subject; (g) reducing oligomeric α-syn level in the brain of a subject; (h) reducing neuropathology and recovery of motor activities in a subject; and the like, wherein the subject is at an early, prodromal stage of their synucleinopathy.

The above-described methods comprise administering a pharmaceutical composition comprising a pharmacologically effective amount of an immunotherapy targeting α-syn (e.g., one or more peptide immunogen construct and/or an antibody, e.g., as described herein) to a subject in need thereof. The amounts and regimens used in the methods can be consistent with the information provided above in the section concerning compositions or as determined to be appropriate by those of skill in the art.

The invention also provides the compositions and kits described herein for use in the prevention, amelioration, inhibition, slowing, or treatment of any of the diseases or conditions described herein.

The following examples illustrate certain features and aspects of the disclosure and are not to be considered as limiting of the scope of the disclosure in any way.

### Examples

Alpha synuclein (α-syn) has a key role in the pathogenesis of Parkinson's disease (PD), Dementia with Lewy Bodies (LBD) and Multiple System Atrophy (MSA). Immunotherapies aiming at neutralising toxic α-syn species are being investigated in the clinic as potential disease modifying therapies for PD and other synucleinopathies. In this study, the effects of active immunisation against α-syn with the UB312 vaccine were investigated in the Thy1SNCA/15 mouse model of PD. Young transgenic and wild type mice received an immunisation regimen over a period of 6 weeks, then observed for an additional 9 weeks. Behavioural assessment was conducted before immunisation and at 15 weeks after the first dose.

UB312 immunisation prevented the development of motor impairment in the wire test and challenging beam test, which was associated with reduced levels of α-syn oligomers in the cerebral cortex, hippocampus and striatum of Thy1SNCA/15 mice. UB312 immunotherapy resulted in a significant reduction of the α-syn load in the colon, accompanied by a reduction in enteric glial cell reactivity in the colonic ganglia.

Our results demonstrate that immunisation with UB312 prevents functional deficits and both central and peripheral pathology in Thy1SNCA/15 mice.

### Materials and Methods

### Animals

Thy1SNCA/15 mice (Stock No. 017682) were obtained from the Jackson Laboratory (Bar Harbour, Maine, USA) and rederived at the University of Southampton to establish and maintain colonies. The Thy1SNCA/15 mice overexpress 1-2 copies of the gene encoding human wild type α-syn that is driven by the mouse thymus cell antigen 1 (Thy1) promoter (Choi et al., Nat. Commun. 11(1):1386, 2020). Thy1SNCA/15 mice demonstrate widespread α-syn expression that appears mainly synaptic with no reported LB-like aggregates or phosphorylated α-syn up to 10 months of age (Rabl et al., BMC Neurosci. 1822, 2017; Choi et al., Nat. Commun. 11(1):1386, 2020). Non-transgenic (C57BL/6J background) littermate mice were used as controls. No behavioural studies have been conducted to date in Thy1SNCA/15 mice

All mice were housed in groups of 5-10, kept under a standard 12-hour light/dark cycle and fed a standard RM1 chow diet (SDS, UK) and water ad libitum. All procedures were carried out in accordance with animal care guidelines stipulated by the United Kingdom Animals (Scientific Procedures) Act 1986, Home Office license.

Vaccination of Mice with UB312 and Antibody Titres

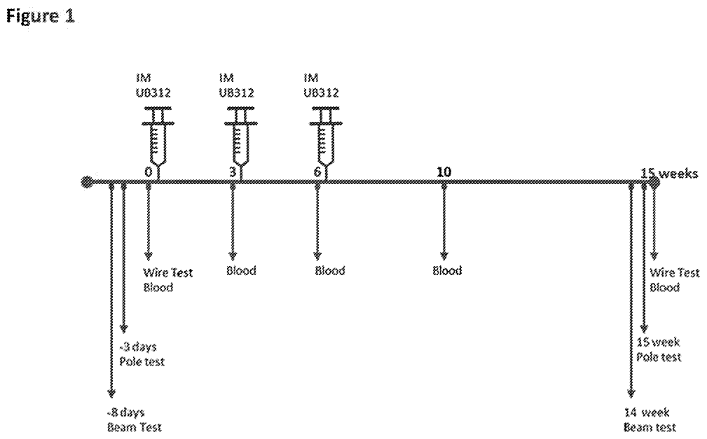

The immunisation regime is summarised in FIG. 1. 10-week-old Thy1SNCA/15 were administered 3 intramuscular injections (3 weeks apart) of either UB312 (40 μg per injection, n=29) or the adjuvant (Adju-Phos® and CpG1) (n=27). 10-week-old non-transgenic C57BL/6J mice also received equivalent immunisations with adjuvant (n=22). Sera were collected before each injection, and at week 10 and 15 after the primary injection for antibody titre analysis. Antibody titres were measured using an anti-α-syn enzyme immunoassay (EIA) kit (United Biomedical, Inc.) which employs a synthetic target peptide immunosorbent against the region K97-D135 of α-syn. UB312: UBITh1-εK-KKK-α-synuclein 126-135 (SEQ ID NO: 112; UBITh1-εk-kkk-EMPSEEGYQD).

Fifteen weeks after the prime injection mice were terminally anaesthetised with pentobarbitone (200 mg/kg) and perfused for immunohistochemistry (Tg-UB-312, n=12; Tg-Adj, n=11; WT-Adj, n=9) or biochemical analysis (Tg-UB-312, n=17; Tg-Adj, n=16; WT-Adj, n=13). For immunohistochemical analysis, mice were intracardially perfused with PBS (0.01 M) followed by 4% Paraformaldehyde (PFA) (in 0.01 M PBS, pH 7.4). The brains and intestines (duodenum and proximal colon) were dissected out and submersed in 4% PFA for a further 4 hours, and subsequently transferred to 30% sucrose for cryoprotection. For western blot analysis, mice were perfused with ice cold PBS (0.01 M) and the cortex, hippocampus, and striatum were immediately dissected out on ice cold PBS and snap-frozen on dry ice for further processing.

### Behaviour Testing

Prior to immunisation and at 15 weeks after the first immunisation dose, mice were subject to three different behavioural tests, each performed on separate days including habituation periods such that there was no overlap of behaviour tests on any day. The order of the tests and habituation periods were kept the same before and after treatment (Tg-UB-312, n=29; Tg-Adj, n=27; WT-Adj, n=22). The assessor was blinded to the animal's treatment status.

### Challenging Beam Traversal Test

Mice were trained to traverse a 1 m long beam composed of 4 equal segments that become narrower towards the end (3.5, 2.5, 1.5, 0.5 cm width). Mice were placed on the wide end of the beam and encouraged to traverse the beam to a clean cage on the opposite side. Mice were given 5 trials per day over 3 days followed by a test day. On the test day, a 1 cm² wire mesh was placed over the beam segments and the mice were allowed to freely traverse the beam for 5 trials. Video recordings were analysed for each trial and the number of errors were recorded. An error was considered if the mouse was moving forward and one of their feet slipped halfway down the wire mesh. The average number of errors over the 5 trials was calculated.

### Pole Test

The pole test consists of a vertical pole (1.5 cm in diameter and 55 cm high) secured in a clean cage. Mice were placed with their head oriented upward on the side of the pole and the times to reorient themselves 180° facing down and descend the pole were recorded. Each mouse underwent 3 days of habituation, up to 5 trials per session followed by a test day.

### Wire Hanging Test

Mice were only subject to one trial on the wire test before and after immunotherapy. Mice were placed hanging upside down on a 6 mm thick wire loop (20 cm in diameter) that could freely rotate on a pivot. Inappropriate behaviour such as balancing on top of the wire, or deliberate jumping off the wire was discouraged and the trial discarded or repeated. The total time to fall off the wire was recorded with a cut-off of 5 minutes.

### Immunohistochemistry

Sagittal sections of 20 μm thickness from brain (1800 μm from midline) or intestines were cut using a Leica Cryostat. α-Syn was detected using immunofluorescence. Briefly, tissue sections were rehydrated in 0.01 M PBS (Sigma, 1002795531) and blocked in 15% normal goat serum (Fisher Scientific, 1002817944) for 1 hour. The sections were incubated overnight at 4° C. in the anti-α-syn antibody, MJFR1 (1:2000, Abcam, ab138501) in 0.01 M PBS, 0.1% Triton X [1001466726, ThermoFisher]. The sections were then incubated at room temperature (RT) in an Alexa-Fluor 555 conjugated goat-anti-rabbit secondary antibody (Molecular Probes life technologies). Sections were counterstained with DAPI and mounted in Mowiol and Citifluor (ThermoFisher).

To analyse the inflammatory status in the brain and gut, markers for astrocytes (GFAP, 1:400, Dako), microglia (Iba1, 1:400, Wako, 019-19741), T-cells (CD3 (KT3), 1200, BioRad, MCA500G), and endothelial activation (ICAM1, 1:200, Biolegend, 116101) were selected. Endogenous peroxidase activity was quenched with 3% H₂O₂ (H1009-500 ml, Sigma Aldrich) for 10 minutes. Heat induced antigen retrieval was performed for Iba1 staining by heating the tissue in citrate buffer (15 mM Tris sodium citrate [101578237, Sigma Aldrich], 0.1% tween, pH6 [P1379, Sigma Aldrich]) using a Panasonic 800W microwave at medium heat for 25 minutes. Non-specific binding sites were blocked with 15% normal goat serum (Fisher Scientific) for 1 hour. The tissue was then incubated overnight at 4° C. with primary antibody in 0.01M PBS, 0.1% triton X. The tissue was then incubated for 1 hour in biotinylated secondary antibodies at RT. Tissue was incubated in Avidin biotin complex (ABC) for 1 hour at RT (PK-6100 Vectastain ABC kit). Development of the chromogen was performed using Nickel DAB. Prior to mounting in Distyrene Plasticizer Xylene (DPX, 12658646 Fisher Scientific), the tissue was dehydrated for 2 minutes each in IMS 50%, 70%, 95%, 100%, counterstained with eosin and incubated in Xylene for 5 minutes.

### Western Blot

Tissue samples were homogenised on ice using a Kontes pellet pestle homogeniser in 10% W/V Radioimmunoprecipitation assay (RIPA) buffer (ThermoFisher, 89901) with HALT protease and phosphatase inhibitor cocktail (ThermoScientific, 78442). The homogenate was centrifuged at 14000 rpm, 4° C. in an Eppendorf 5417 R benchtop centrifuge. The pellet was discarded and the supernatant retained for analysis. The protein concentration of each supernatant was determined using Pierce bovine serum albumin (BSA) assay kit (ThermoFisher, 23227) following the manufacturer's instructions.

A Mini-PROTEIN Tetra vertical electrophoresis cell (BioRad; 1568004) was used for the separation of protein from brain homogenates. 1 mm thick polyacrylamide gels were prepared for either denaturing conditions or native conditions.

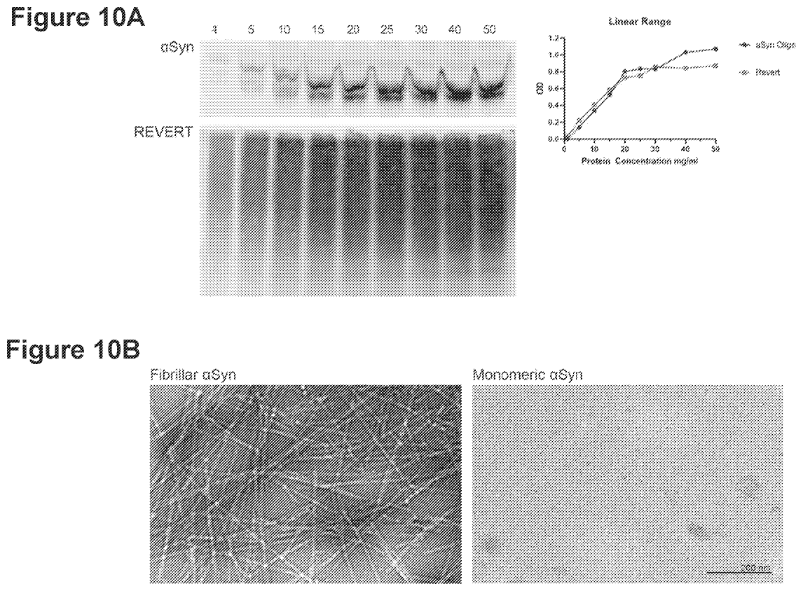

For native polyacrylamide gel electrophoresis (PAGE), brain homogenates were diluted in 4× Laemmli sample buffer (BioRad, 1620112) and 20 μg protein loaded into a 10 or 12% native gel. Pure monomeric α-syn (FIG. 10A) was run alongside the brain homogenate as a molecular weight marker. Protein concentrations for loading were determined from the linear range of the antibodies used (FIG. 10A). Electrophoresis was conducted at 100-150 V in Laemmli buffer (192 mM Glycine [Sigma Aldrich, G8898], 25 mM Tris Base [ThermoFisher, 10103203]) for 2 hours. Semi-dry transfer was conducted using Trans Blot turbo system (BioRad, 1704150) and Mini transfer kit (BioRad, 1704270). Protein was transferred to 0.2 μm nitrocellulose membranes at 2.5 V, 2 A and 15 minutes. Membranes were blocked with 3% Bovine serum albumin (BSA) (Sigma Aldrich, 102052095) for 1 hour at RT. After washing the membranes 3×5 minutes in Tris buffered saline (TBS) (0.25 M Tris Base, 1.5 M NaCl, pH 7.2), 0.1% tween20 (Sigma, P1379), they were incubated overnight at 4° C. in MJFR1 (1:5000; ab138501, Abcam). Revert 700 total protein stain (LiCor, 926-11015) was applied prior to blocking in BSA for normalisation of protein loading.

### Image Analysis and Statistics

Immunoblots were imaged on a LiCor Odyssey Fc scanner and analysed using Image Studio Lite V5.2. Immunoreactive α-syn bands were normalised to GAPDH for SDS-PAGE and Revert for native PAGE. Immunostained tissue sections processed for fluorescence microscopy were visualised, and images captured, at 20× using an SP8 confocal-laser scanning microscope (Milton Keys, UK). DAB immunostained tissue sections were scanned for analysis at ×20 using an Olympus VS110 high throughput Virtual Microscopy System. Images (each 0.16 mm²) were captured from the scanned image using Olympus VS software. For each marker studied, the percentage area of immunoreactivity over 2 consecutive sections per animal was calculated using FIJI software. The average percentage area was calculated for each brain region and statistical analysis was conducted using GraphPad Prism software. A two-way analysis of variance (ANOVA) was used for behavioural analysis with Bonferroni corrections for post hoc multiple comparisons. T-tests were conducted unless otherwise specified for analysing α-syn immunoreactivity in western blots and immunohistochemistry. One-way ANOVA was used for analysing inflammatory markers. Post hoc analysis was conducted with Bonferroni corrections for multiple comparison analysis where applicable. Differences were considered as significant when p<0.05. Numbers (n) refer to the number of mice used for each experiment.

### Results

### Antibody Titres

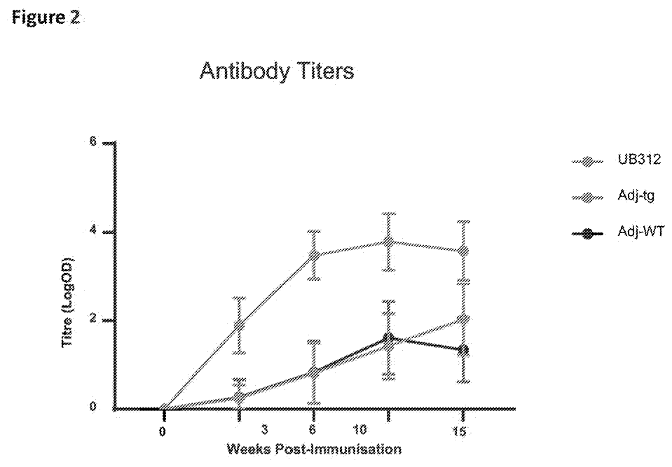

All transgenic mice produced high levels of anti-α-syn97-135 antibody titres after the first injection. Titre levels rose rapidly in the first 6 weeks, peaked between week 6-10 and remained stable for the rest of the 15-week study period (FIG. 2). Unexpectedly, some Wt and transgenic mice administered adjuvant also produced background antibody titres, but these were 2-3 orders of magnitude lower than UB312 induced titres.

### UB312 Immunisation Improves Motor Performance

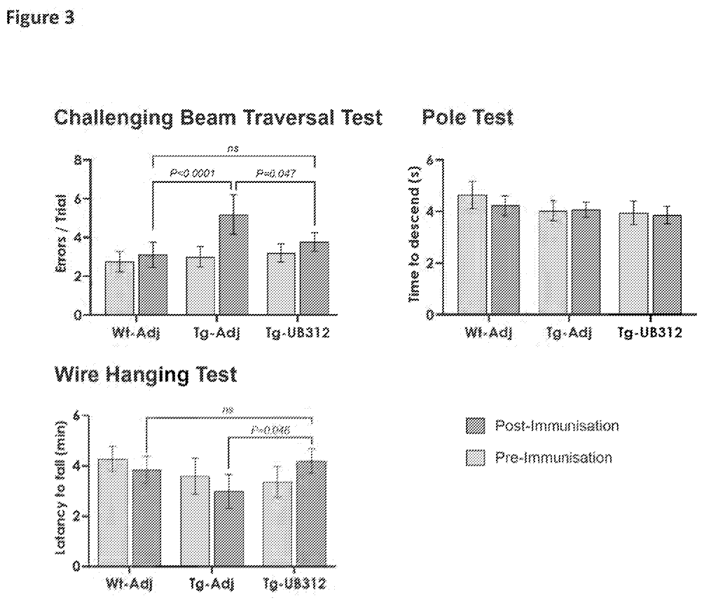

The effect of UB312 immunotherapy on functional outcome in Thy1SNCA/15 mice was investigated using three behavioural tests designed to assess motor function. These included the wire-hanging test to measure grip strength, the challenging beam test for sensorimotor performance, and the pole test for voluntary motor control (Fleming et al., J. Neurosci. 24:9434-9440, 2004). At 10 weeks of age, prior to the commencement of immunotherapy, Thy1SNCA/15 mice did not show any difference in motor performance compared to Wt mice in any of the tests, as shown in FIG. 3. The motor performance of Thy1SNCA/15 mice deteriorated with age in the beam and wire test (26 weeks of age) and this deterioration was prevented with 15 weeks of UB312 immunotherapy.

In the challenging beam traversal test, two-way ANOVA revealed a significant effect of age (F_{(1,83)}=22.46, p<0.0001) and treatment (F_{(2,83)}=5.72, p=0.0047) on the number of foot errors. Post hoc analysis of multiple comparisons showed that the control group of Thy1SNCA/15 mice receiving adjuvant made significantly more errors per trial at 6 months of age compared to 10 weeks (P<0.0001) and compared to 6-month-old Wt mice (Wt-Adj: 3.1, Tg-Adj: 5.1; p<0.0001). The number of errors per trial was not significantly different between Wt mice and UB312 treated Thy1SNCA/15 mice (p=0.38).

In the wire hanging test, a significant effect of treatment on the age-related decline seen in the in Thy1SNCA/15 mice receiving adjuvant was observed (F_{(2,80)}=4.03, P=0.022). Post hoc analysis indicated a trend in reduced latency to fall time in the control group of adjuvant-treated Thy1SNCA/15 mice compared to Wt mice (Wt-Adj: 3.85, Tg-Adj: 2.98; p=0.102). This was significantly lower than UB312 treated Thy1SNCA/15 mice (Tg-Adj: 2.98, Tg-UB312: 4.2; p=0.0095). There was no significant difference between Wt mice and UB312 treated Thy1SNCA/15 mice at the end of the treatment period (Wt-Adj: 3.85, Tg-UB312: 4.20; p>0.99).

For the pole test, the time taken for mice to perform a turn and descend the pole was similar between Thy1SNCA/15 mice and Wt mice with no effect of age (F_{(1,64)}=0.156, p=0.6941) or treatment (F_{(2,64)}=2.688, p=0.076) on motor performance.

UB312 Immunisation Reduces α-syn Oligomers in the Brain

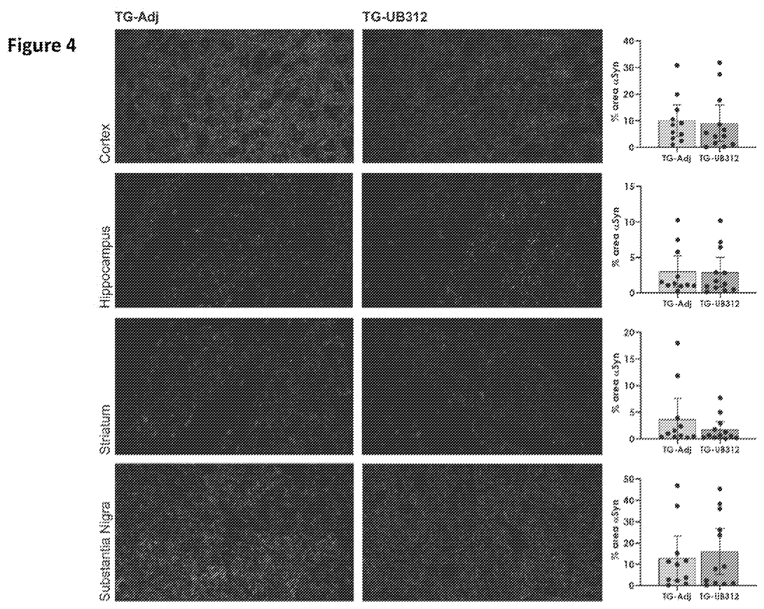

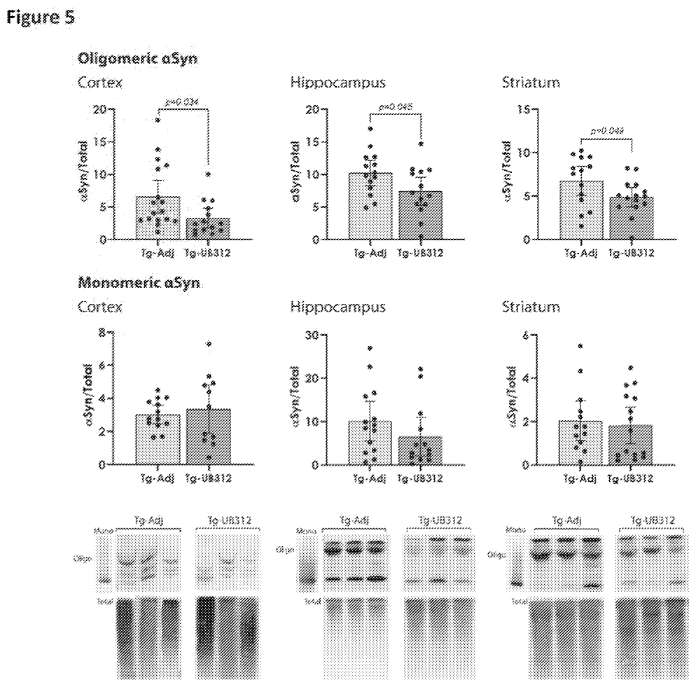

At completion of the 15-week treatment period, 6-month-old mice were anesthetised and tissues collected to assess the effects of UB312 immunotherapy on α-syn-mediated pathology. α-Syn pathology was analysed by immunohistochemistry and western blot using an MJFR1 anti-α-syn antibody, which is specific for human α-syn overexpressed by the Thy1SNCA/15 mice. As expected, Wt mice showed no immunoreactivity for human α-syn and were not included in the quantitative analysis. In Thy1SNCA/15 mice, immunohistochemical staining of brain sections for α-syn showed a widespread granular or punctate pattern in grey matter, consistent with a synaptic location. α-Syn inclusions such as Lewy bodies could not be detected in the brain of 6-month-old Thy1SNCA/15 mice. Quantitative analysis of the percentage area covered by α-syn immunoreactivity in each region of interest (cortex, striatum, hippocampus, substantia nigra, and cerebellum; FIG. 4) did not show any difference between UB312 and adjuvant treated mice. Similarly, the total levels of α-syn detected by western blot analysis (FIG. 5) did not show any difference between UB312 and adjuvant treated mice. In order to investigate whether UB312 specifically reduced higher molecular weight α-syn oligomers, native non-denaturing western blots were performed. Pure monomeric α-syn was used as a molecular weight marker and corresponded to the lowest band in the gels. The results are presented in FIG. 5 and show that UB312 significantly reduced α-syn oligomers but not monomers in the hippocampus by 27.8% (p=0.049), striatum by 27.9% (p=0.045) and the cortex by 49.8% (p=0.035) of Thy1SNCA/15 mice compared to control Thy1SNCA/15 mice administered adjuvant.

UB312 does not Induce Widespread Glial Cell Reaction

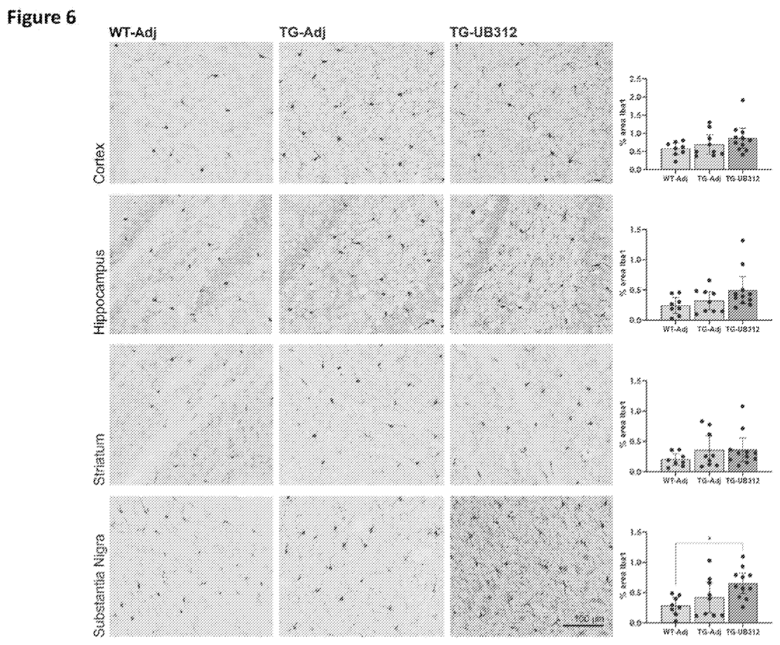

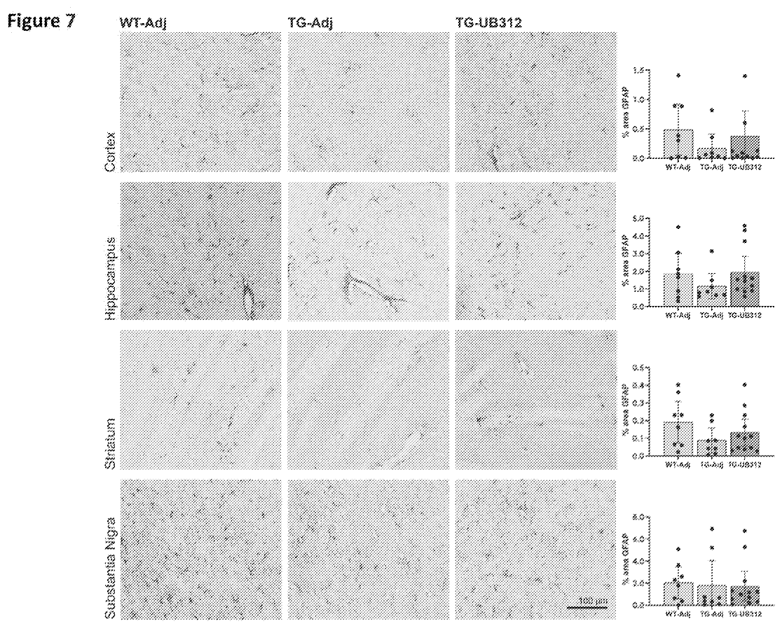

Immunohistochemistry was performed on adjacent tissue sections for glial markers microglia (Iba1) and astrocytes (GFAP). FIG. 6 shows representative images of Iba1 and FIG. 7 shows GFAP immunostaining in each brain region (cortex, hippocampus, striatum, and substantia nigra). One-way ANOVA analysis of Iba1 and GFAP immunoreactivity showed no difference between each group, with the exception of the SN (F_{(2,25)}=4.989) which showed significantly increased Iba1 immunostaining in UB312 treated Thy1SNCA/15 mice compared to control adjuvant treated Thy1SNCA/15 or Wt mice (Wt-Adj: 0.29%, Tg-UB312: 0.65%; p=0.015). Iba1 and GFAP immunoreactivity were comparable between adjuvant treated Thy1SNCA/15 mice and Wt mice across all brain regions.

UB312 does not Induce T Cell Infiltration

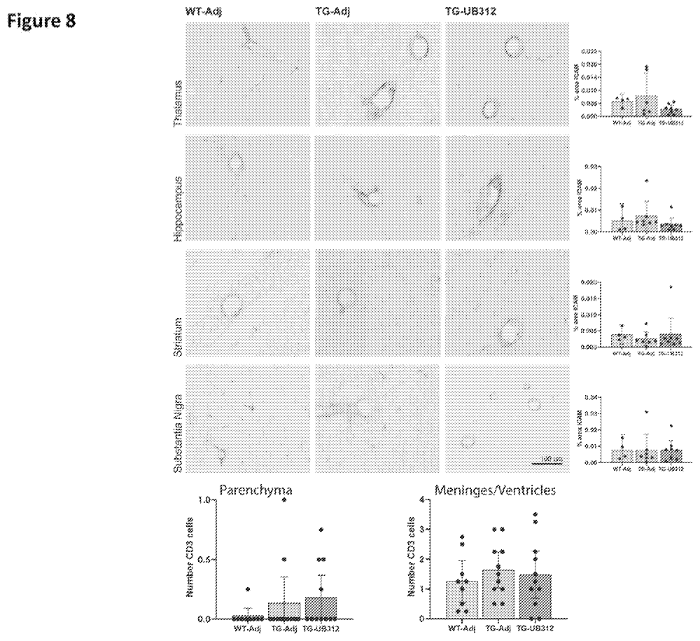

The effect of UB312 treatment on T-cell infiltration was examined by counting the number of parenchymal CD3 positive T-cells over three consecutive 20 μm thick brain sections. The majority of brain sections were negative for CD3 T-cells and there was no increase in T-cell numbers in UB312 treated Thy1SNCA/15 mice (FIG. 8). In order to assess the activation state of the endothelia, ICAM1 immunoreactivity on cerebral endothelial cells was quantified. ICAM1 is expressed on endothelial cells and is upregulated during inflammation to facilitate T cell extravasation. The results are presented in FIG. 8 and show no difference in ICAM1 immunoreactivity between UB312 and adjuvant treated Thy1SNCA/15 or Wt mice.

### UB312 Reduces α-Syn and Enteric Glial Cell Activation in the Colon

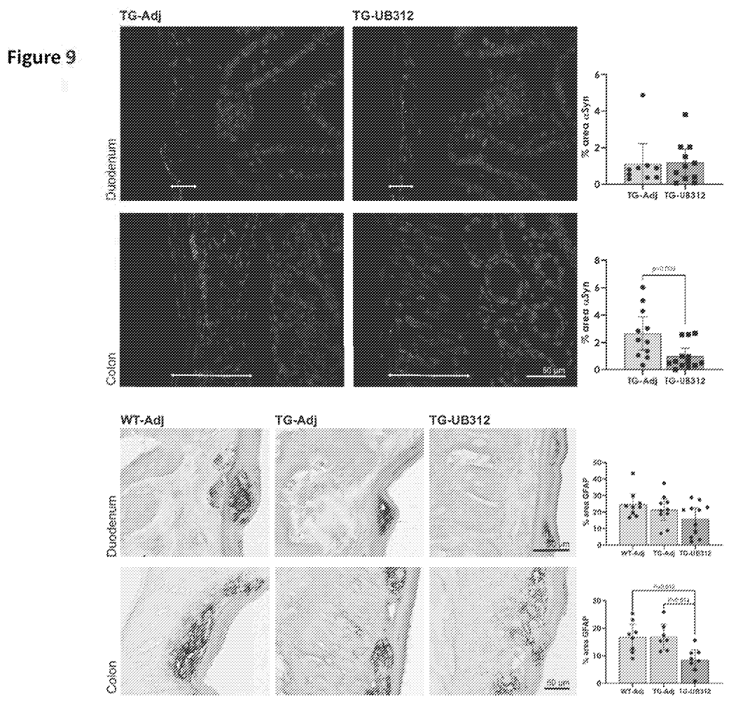

Gastrointestinal (GI) dysfunction is a common prodromal feature of PD, and LBs have been identified in colonic biopsies of PD patients. Thy1SNCA/15 mice display α-syn accumulation in the nerve fibres and synapses of the muscularis layer of the gut wall at 10 weeks of age (FIG. 9). Two-tailed t-test of the percentage α-syn immunoreactivity in the gut wall showed a significant decrease in UB312 treated Thy1SNCA/15 mice when compared to adjuvant controls in the colon (Tg-Adj: 2.65%, Tg-UB312: 0.98%; p=0.0093) but not in the duodenum (Tg-Adj: 1.12%, Tg-UB312: 1.18%; p=0.91).

The pattern of glial cell reactivity in the gut was investigated using a marker for activation of ganglionic enteric glial cells (GFAP). FIG. 9 presents representative images of GFAP immunostaining and subsequent quantification of GFAP immunoreactivity within the myenteric ganglia. One-way-ANOVA revealed a significant treatment effect on GFAP expression (F_{(2,20)}=7.007; p=0.0049). UB312 immunotherapy in Thy1SNCA/15 mice significantly reduced the levels of GFAP expression in the colonic myenteric ganglia when compared to adjuvant treated Thy1SNCA/15 mice (Tg-Adj: 16.86%, Tg-UB312: 8.47%; P=0.014). There was no difference in GFAP immunoreactivity between control groups of Wt and Thy1SNCA/15 adjuvant treated mice (Wt-Adj: 16.73, Tg-Adj: 16.86; p>0.99), whereas UB312 treated Thy1SNCA/15 mice showed a significant reduction in GFAP when compared to Wt mice (Wt-Adj: 16.73%, Tg-Adj: 8.47%; p>0.012). There was no difference in GFAP expression between treatment groups in the duodenum.

TABLE 1
Amino Acid sequences of α-syn and fragments thereof

| | SEQ ID | |
|---|---|---|
| Amino Acid positions | NO: | Sequence |
| α-Synuclein 1-140 | 1 | MDVEM KGLSK AKEGV VAAAE KIKQG VAEAA GKTKE GVLYV GSKTK EGVVH |
| | | GVATV AEKTK EQVIN VGGAV VIGVT AVAQK TVEGA GSIAA ATGEV KKDQL |
| | | GKNEE GAPQE GILED MPVDP DNEAY EMPSE EGYQD YEPEA |
| α-Synuclein 80-140 | 3 | KTVEG AGSIA AATGF VKKDQ LGKNE EGAPQ EGILE DMPVD PDNEA YEMPS |
| | | EEGYQ DYEPE A |
| α-Synuclein 85-140 | 4 | AGSIA AATGF VKKDQ LGKNE EGAPQ EGILE DMPVD PDNEA YEMPS EEGYQ |
| | | DYEPEA |
| α-Synuclein 91-140 | 5 | ATGFV KKDQL GKNEE GAPQE GILED MPVDP DNEAY EMPSE EGYQD YEPEA |
| α-Synuclein 101-140 | 6 | GKNEE GAPQE GILED MPVDP DNEAY EMPSE EGYQD YEPEA |
| α-Synuclein 111-140 | 7 | GILED MPVDP DNEAY EMPSE EGYQD YEPEA |
| α-Synuclein 121-140 | 8 | DNEAY EMPSE EGYQD YEPEA |
| α-Synuclein 126-140 | 9 | EMPSE EGYQD YEPEA |
| α-Synuclein 97-135 | 10 | KDQLG KNEEG APQEG ILEDM PVDPD NEAYE MPSEE GYQD |
| α-Synuclein 101-135 | 11 | GKNEE GAPQE GILED MPVDP DNEAY EMPSE EGYQD |
| α-Synuclein 111-135 | 12 | GILED MPVDP DNEAY EMPSE EGYQD |
| α-Synuclein 121-135 | 13 | DNEAY EMPSE EGYQD |
| α-Synuclein 123-135 | 14 | EAYEM PSEEG YQD |
| α-Synuclein 126-135 | 15 | EMPSE EGYQD |
| α-Synuclein 101-132 | 16 | GKNEE GAPQE GILED MPVDP DNEAY EMPSE EG |
| α-Synuclein 111-132 | 17 | GILED MPVDP DNEAY EMPSE EG |
| α-Synuclein 80-89 | 18 | KTVEG AGSIA |
| α-Synuclein 81-90 | 19 | TVEGA GSIAA |
| α-Synuclein 82-91 | 20 | VEGAG SIAAA |
| α-Synuclein 83-92 | 21 | EGAGS IAAAT |
| α-Synuclein 84-93 | 22 | GAGSI AAATG |
| α-Synuclein 85-94 | 23 | AGSIA AATGE |
| α-Synuclein 86-95 | 24 | GSIAA ATGFV |
| α-Synuclein 87-96 | 25 | SIAAA TGFVK |
| α-Synuclein 88-97 | 26 | IAAAT GFVKK |
| α-Synuclein 89-98 | 27 | AAATG FVKKD |
| α-Synuclein 90-99 | 28 | AATGF VKKDQ |
| α-Synuclein 91-100 | 29 | ATGFV KKDQL |
| α-Synuclein 92-101 | 30 | TGFVK KDQLG |
| α-Synuclein 93-102 | 31 | GFVKK DQLGK |
| α-Synuclein 94-103 | 32 | FVKKD QLGKN |
| α-Synuclein 95-104 | 33 | VKKDQ LGKNE |
| α-Synuclein 96-105 | 34 | KKDQL GKNEE |
| α-Synuclein 97-106 | 35 | KDQLG KNEEG |
| α-Synuclein 98-107 | 36 | DQLGK NEEGA |
| α-Synuclein 99-108 | 37 | QLGKN EEGAP |
| α-Synuclein 100-109 | 38 | LGKNE EGAPQ |
| α-Synuclein 101-110 | 39 | GKNEE GAPQE |
| α-Synuclein 102-111 | 40 | KNEEG APQEG |
| α-Synuclein 103-112 | 41 | NEEGA PQEGI |
| α-Synuclein 104-113 | 42 | EEGAP QEGIL |
| α-Synuclein 105-114 | 43 | EGAPQ EGILE |
| α-Synuclein 106-115 | 44 | GAPQE GILED |
| α-Synuclein 107-116 | 45 | APQEG ILEDM |
| α-Synuclein 108-117 | 46 | PQEGI LEDMP |
| α-Synuclein 109-118 | 47 | QEGIL EDMPV |
| α-Synuclein 110-119 | 48 | EGILE DMPVD |
| α-Synuclein 111-120 | 49 | GILED MPVDP |
| α-Synuclein 112-121 | 50 | ILEDM PVDPD |
| α-Synuclein 113-122 | 51 | LEDMP VDPDN |
| α-Synuclein 114-123 | 52 | EDMPV DPDNE |
| α-Synuclein 115-124 | 53 | DMPVD PDNEA |
| α-Synuclein 116-125 | 54 | MPVDP DNEAY |
| α-Synuclein 117-126 | 55 | PVDPD NEAYE |
| α-Synuclein 118-127 | 56 | VDPDN EAYEM |
| α-Synuclein 119-128 | 57 | DPDNE AYEMP |
| α-Synuclein 120-129 | 58 | PDNEA YEMPS |
| α-Synuclein 121-130 | 59 | DNEAY EMPSE |
| α-Synuclein 122-131 | 60 | NEAYE MPSEE |
| α-Synuclein 123-132 | 61 | EAYEM PSEEG |
| α-Synuclein 124-133 | 62 | AYEMP SEEGY |
| α-Synuclein 125-134 | 63 | YEMPS EEGYQ |
| α-Synuclein 126-135 | 64 | EMPSE EGYQD |
| α-Synuclein 127-136 | 65 | MPSEE GYQDY |
| α-Synuclein 128-137 | 66 | PSEEG YQDYE |
| α-Synuclein 129-138 | 67 | SEEGY QDYEP |
| α-Synuclein 130-139 | 68 | EEGYQ DYEPE |
| α-Synuclein 131-140 | 69 | EGYQD YEPEA |

TABLE 2
Amino Acid Sequences of Pathogen Protein Derived Th Epitopes Including
Idealized Artificial Th Epitopes for Employment in the Design of α-Syn
Peptide Immunogen Constructs

| | SEQ ID | |
|---|---|---|
| Description | NO: | Sequence |
| Clostridium tetani1 Th | 70 | KKQYIKANSKFIGITEL |
| MvF1 Th | 71 | LSEIKGVIVHRLEGV |
| Bordetella pertussis Th | 72 | GAYARCPNGTRALTVAELRGNAEL |
| Clostridium tetani2 Th | 73 | WVRDIIDDFTNESSQKT |
| Diphtheria Th | 74 | DSETADNLEKTVAALSILPGHGC |
| Plasmodium falciparum Th | 75 | DHEKKHAKMEKASSVFNVVNS |
| Schistosoma mansoni Th | 76 | KWFKTNAPNGVDEKHRH |
| Cholera Toxin Th | 77 | ALNIWDRFDVFCTLGATTGYLKGNS |
| MvF2 Th | 78 | ISEIKGVIVHKIEGI |
| KKKMvF3 Th | 79 | KKKISISEIKGVIVHKIEGILF |
| | | T RT TR T |
| HBsAg1 Th | 80 | KKKLFLLTKLLTLPQSLD |
| | | RRRIKII RII I L IR |
| | | VRVV VV V I V |
| | | F FF FF F V F |
| | | F |
| MvF4 Th (UBITh ®3) | 81 | ISISEIKGVIVHKIETILF |
| | | T RT TR |
| HBsAg2 Th | 82 | KKKIITITRIITIPQSLD |
| | | FFLL L ITTI |
| MvF5 Th (UBITh ®1) | 83 | ISITEIKGVIVHRIETILF |
| HBsAg3 Th (UBITh ®2) | 84 | KKKIITITRIITIITTID |
| Influenza MP1_1 Th | 85 | FVFTLTVPSER |
| Influenza MP1_2 Th | 86 | SGPLKAEIAQRLEDV |
| Influenza NSP1 Th | 87 | DRLRRDQKS |
| EBV BHRF1 Th | 88 | AGLTLSLLVICSYLFISRG |
| Clostridium tetani TT1 Th | 89 | QYIKANSKFIGITEL |
| EBV EBNA-1 Th | 90 | PGPLRESIVCYFMVELQTHI |
| Clostridium tetani TT2 Th | 91 | FNNFTVSFWLRVPKVSASHLE |
| Clostridium tetani TT3 Th | 92 | KFIIKRYTPNNEIDSF |
| Clostridium tetani TT4 Th | 93 | VSIDKFRIFCKALNPK |
| EBV CP Th | 94 | VPGLYSPCRAFFNKEELL |
| HCMV IE1 Th | 95 | DKREMWMACIKELH |
| EBV GP340 Th | 96 | TGHGARTSTEPTTDY |
| EBV BPLF1 Th | 97 | KELKRQYEKKLRQ |
| EBV EBNA-2 Th | 98 | TVFYNIPPMPL |

TABLE 3
Amino Acid Sequences of α-Syn Peptide Immunogen Constructs

| | Seq | |
|---|---|---|
| | ID | |
| Peptide Description | NO: | Sequence |
| UBITh3-ϵK-KKK-α-Synuclein 126-140 | 99 | UBITh3-ϵk-kkk-EMPSEEGYQDYEPEA |
| UBITh3-ϵK-KKK-α-Synuclein 121-140 | 100 | UBITh3-ϵk-kkk-DNEAYEMPSEEGYQDYEPEA |
| UBITh3-ϵK-KKK-α-Synuclein 111-140 | 101 | UBITh3-ϵk-kkk-GILEDMPVDPDNEAYEMPSEEGYQDYEPEA |
| UBITh3-ϵK-KKK-α-Synuclein 101-140 | 102 | UBITh3-ϵk-kkk-GKNEEGAPQEGILEDMPVDPDNEAYEMPSEEGYQDYEPEA |
| UBITh1-ϵK-KKK-α-Synuclein 101-140 | 103 | UBITh1-ϵk-kkk-GKNEEGAPQEGILEDMPVDPDNEAYEMPSEEGYQDYEPEA |
| UBITh2-ϵK-KKK-α-Synuclein 101-140 | 104 | UBITh2-ϵk-kkk-GKNEEGAPQEGILEDMPVDPDNEAYEMPSEEGYQDYEPEA |
| UBITh3-ϵK-KKK-α-Synuclein 91-140 | 105 | UBITh3-ϵk-kkk- |
| | | ATGFVKKDQLGKNEEGAPQEGILEDMPVDPDNEAYEMPSEEGYQDYEPEA |
| UBITh3-ϵK-KKK-α-Synuclein 85-140 | 106 | UBITh3-ϵk-kkk- |
| | | AGSIAAATGFVKKDQLGKNEEGAPQEGILEDMPVDPDNEAYEMPSEEGYQDYEPEA |
| UBITh1-ϵK-KKK-α-Synuclein 121-135 | 107 | UBITh1-ϵk-kkk-DNEAYEMPSEEGYQD |
| UBITh1-ϵK-KKK-α-Synuclein 111-135 | 108 | UBITh1-ϵk-kkk-GILEDMPVDPDNEAYEMPSEEGYQD |
| UBITh1-ϵK-KKK-α-Synuclein 101-135 | 109 | UBITh1-ϵk-kkk-GKNEEGAPQEGILEDMPVDPDNEAYEMPSEEGYQD |
| UBITh1-ϵK-KKK-α-Synuclein 97-135 | 110 | UBITh1-ϵk-kkk-KDQLGKNEEGAPQEGILEDMPVDPDNEAYEMPSEEGYQD |
| UBITh1-ϵK-KKK-α-Synuclein 123-135 | 111 | UBITh1-ϵk-kkk-EAYEMPSEEGYQD |
| UBITh1-ϵK-KKK-α-Synuclein 126-135 | 112 | UBITh1-ϵk-kkk-EMPSEEGYQD |
| UBITh1-ϵK-KKK-α-Synuclein 111-132 | 113 | UBITh1-ϵk-kkk-GILEDMPVDPDNEAYEMPSEEG |
| UBITh1-ϵK-KKK-α-Synuclein 101-132 | 114 | UBITh1-ϵk-kkk-GKNEEGAPQEGILEDMPVDPDNEAYEMPSEEG |
| UBITh1-ϵK-KKK- Mouse counterpart | 115 | UBITh1-ϵk-kkk-GILEDMPVDPGSEAYEMPSEEG |
| α-Synuclein 111-132 | | |
| UBITh3-ϵK-KKK-α-Synuclein 126-135 | 116 | UBITh3-ϵk-kkk-EMPSEEGYQD |
| UBITh3-ϵK-KKK-α-Synuclein 111-132 | 117 | UBITh3-ϵk-kkk-GILEDMPVDPDNEAYEMPSEEG |
| UBITh1-ϵK-α-Synuclein 126-135 | 118 | UBITh1-ϵk-EMPSEEGYQD |
| UBITh1-ϵK-α-Synuclein 111-132 | 119 | UBITh1-ϵk-GILEDMPVDPDNEAYEMPSEEG |
| UBITh2-ϵK-α-Synuclein 126-135 | 120 | UBITh2-ϵk-EMPSEEGYQD |
| UBITh2-ϵK-α-Synuclein 111-132 | 121 | UBITh2-ϵk-GILEDMPVDPDNEAYEMPSEEG |
| Clostridium tetani1 Th-ϵK-α-Syn | 122 | KKQYIKANSKFIGITEL-ϵk-GILEDMPVDPDNEAYEMPSEEG |
| 111-132 | | |
| MvF1 Th-ϵK-α-Synuclein 111-132 | 123 | LSEIKGVIVHRLEGV-ϵk-GILEDMPVDPDNEAYEMPSEEG |
| Bordetella pertussis Th-ϵK-α-Syn | 124 | GAYARCPNGTRALTVAELRGNAEL-ϵk-GILEDMPVDPDNEAYEMPSEEG |
| 111-132 | | |
| Clostridium tetani2 Th-ϵK-α-Syn | 125 | WVRDIIDDETNESSQKT-ϵk-GILEDMPVDPDNEAYEMPSEEG |
| 111-132 | | |
| Diphtheria Th-ϵK-α-Syn 111-132 | 126 | DSETADNLEKTVAALSILPGHGC-ϵk-GILEDMPVDPDNEAYEMPSEEG |
| Plasmodium falciparum Th-ϵK-α-Syn | 127 | DHEKKHAKMEKASSVENVVNS-ϵk-GILEDMPVDPDNEAYEMPSEEG |
| 111-132 | | |
| Schistosoma mansoni Th-ϵK-α-Syn | 128 | KWFKTNAPNGVDEKHRH-ϵk-GILEDMPVDPDNEAYEMPSEEG |
| 111-132 | | |
| Cholera Toxin Th-ϵK-α-Syn 111-132 | 129 | ALNIWDRFDVFCTLGATTGYLKGNS-ϵk-GILEDMPVDPDNEAYEMPSEEG |
| MvF2 Th-ϵK-α-Syn 111-132 | 130 | ISEIKGVIVHKIEGI-ϵk-GILEDMPVDPDNEAYEMPSEEG |
| KKKMvF3 Th-ϵK-α-Syn 111-132 | 131 | KKKISISEIKGVIVHKIEGILF-ϵk-GILEDMPVDPDNEAYEMPSEEG |
| | | T RT TR T |
| HBsAg1 Th-ϵK-α-Syn 111-132 | 132 | KKKLFLLTKLLTLPQSLD-ϵk-GILEDMPVDPDNEAYEMPSEEG |
| | | RRRIKII RII I L IR |
| | | VRVV VV V I V |
| | | F FF FF F V F |
| | | F |
| HBsAg2 Th-ϵK-α-Syn 111-132 | 133 | KKKIITITRIITIPQSLD-ϵk-GILEDMPVDPDNEAYEMPSEEG |
| | | FELLLITTI |
| Influenza MP1_1 Th-ϵK-α-Syn 111-132 | 134 | FVFTLTVPSER-ϵk-GILEDMPVDPDNEAYEMPSEEG |
| Influenza MP1_2 Th-ϵK-α-Syn 111-132 | 135 | SGPLKAEIAQRLEDV-ϵk-GILEDMPVDPDNEAYEMPSEEG |
| Influenza NSP1 Th-ϵK-α-Syn 111-132 | 136 | DRLRRDQKS-ϵk-GILEDMPVDPDNEAYEMPSEEG |
| EBV BHRF1 Th-ϵK-α-Syn 111-132 | 137 | AGLILSLLVICSYLFISRG-ϵk-GILEDMPVDPDNEAYEMPSEEG |
| Clostridium tetani TT1 Th-ϵK-α-Syn | 138 | QYIKANSKFIGITEL-ϵk-GILEDMPVDPDNEAYEMPSEEG |
| 111-132 | | |
| EBV EBNA-1 Th-ϵK-α-Syn 111-132 | 139 | PGPLRESIVCYFMVELQTHI-ϵk-GILEDMPVDPDNEAYEMPSEEG |
| Clostridium tetani TT2 Th-ϵK-α-Syn | 140 | FNNFTVSFWLRVPKVSASHLE-ϵk-GILEDMPVDPDNEAYEMPSEEG |
| 111-132 | | |
| Clostridium tetani TT3 Th-ϵK-α-Syn | 141 | KFIIKRYTPNNEIDSF-ϵk-GILEDMPVDPDNEAYEMPSEEG |
| 111-132 | | |
| Clostridium tetani TT4 Th-ϵK-α-Syn | 142 | VSIDKFRIFCKALNPK-ϵk-GILEDMPVDPDNEAYEMPSEEG |
| 111-132 | | |
| EBV CP Th-ϵK-α-Syn 111-132 | 143 | VPGLYSPCRAFFNKEELL-ϵk-GI LEDMPVDPDNEAYEMPSEEG |
| HCMV IE1 Th-ϵK-α-Syn 111-132 | 144 | DKREMWMACIKELH-ϵk-GILEDMPVDPDNEAYEMPSEEG |
| EBV GP340 Th-ϵK-α-Syn 111-132 | 145 | TGHGARTSTEPTTDY-ϵk-GILEDMPVDPDNEAYEMPSEEG |
| EBV BPLF1 Th-ϵK-α-Syn 111-132 | 146 | KELKRQYEKKLRQ-ϵk-GILEDMPVDPDNEAYEMPSEEG |
| EBV EBNA-2 Th-ϵK-α-Syn 111-132 | 147 | TVFYNIPPMPL-ϵk-GILEDMPVDPDNEAYEMPSEEG |

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Some embodiments are within the scope of the following numbered paragraphs.

1. A method of preventing, reducing, inhibiting, or slowing the development of one or more motor symptom of a synucleinopathy in a subject in need thereof, the method comprising administering to the subject an effective amount of an immunotherapy targeting alpha-synuclein (α-syn).

2. The method of numbered paragraph 1, wherein the one or more motor symptom of a synucleinopathy is selected from the group consisting of muscle rigidity, bradykinesia, tremor at rest, and postural instability.

3. A method of treating, preventing, reducing, or inhibiting one or more gastrointestinal symptom of a synucleinopathy in a subject in need thereof, the method comprising administering to the subject an effective amount of an immunotherapy targeting α-syn.

4. The method of numbered paragraph 3, wherein the one or more gastrointestinal symptom is selected from the group consisting of: drooling, salivation, dysphagia, nausea, vomiting, dyspepsia, constipation, abdominal pain, gastroparesis, and fecal incontinence.

5. The method of numbered paragraph 3 or 4, wherein the gastrointestinal symptom occurs in the colon of the subject.

6. A method of reducing the level of α-syn in the gastrointestinal tract (e.g., the colon) of a subject in need thereof, the method comprising administering to the subject an effective amount of an immunotherapy targeting α-syn.

7. The method of any one of numbered paragraphs 1 to 6, wherein the subject does not have one or more motor symptom of a synucleinopathy or exhibits only a minimal motor symptom of a synucleinopathy.

8. The method of numbered paragraph 7, wherein the subject does not have one or more motor symptom of a synucleinopathy selected from the group consisting of muscle rigidity, bradykinesia, tremor at rest, and postural instability.

9. The method of any one of numbered paragraphs 1 to 8, wherein the subject has a synucleinopathy at an early, prodromal stage.

10. A method of inducing an immune response to α-syn in a subject, inhibiting α-syn aggregation in a subject, or reducing the amount of α-syn aggregates in a subject, the method comprising administering an effective amount of an immunotherapy targeting α-syn to the subject, wherein the subject has a synucleinopathy at an early, prodromal stage.

11. The method of any one of numbered paragraphs 1 to 10, wherein the synucleinopathy is selected from the group consisting of Parkinson's disease (PD), Parkinson's disease dementia (PDD), dementia with Lewy bodies (DLB), multiple system atrophy (MSA), neuroaxonal dystrophies, and pure autonomic failure (PAF).

12. The method of any one of numbered paragraphs 1 to 11, wherein the immunotherapy comprises a peptide, a protein (e.g., an antibody), a fragment or fusion of a peptide or a protein (e.g., an antibody), or a nucleic acid molecule (e.g., an mRNA or a nucleic acid in a vector) encoding one of said molecules.

13. The method of any one of numbered paragraphs 1 to 12, wherein the immunotherapy comprises a peptide immunogen construct.

14. The method of numbered paragraph 13, wherein the peptide immunogen construct comprises a B cell epitope, a heterologous T cell epitope, and an optional linker.

15. The method of numbered paragraph 14, wherein the B cell epitope induces an immune response against α-syn.

16. The method of numbered paragraph 15, wherein the B cell epitope comprises a peptide of the C-terminal region of an α-syn protein, wherein the peptide optionally is about 10 to about 25 amino acids in length.

17. The method of numbered paragraph 16, wherein the α-syn protein comprises the sequence of SEQ ID NO: 1.

18. The method of any one of numbered paragraphs 14 to 17, wherein the B cell epitope comprises a peptide selected from a sequence of Table 1 (e.g., any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69).

19. The method of any one of numbered paragraphs 14 to 18, wherein the heterologous T cell epitope is derived from a pathogenic protein.

20. The method of any one of numbered paragraphs 14 to 19, wherein the heterologous T cell epitope comprises a sequence selected from a sequence of Table 2.

21. The method of any one of numbered paragraphs 14 to 20, wherein the peptide comprises a heterologous spacer or linker between the B cell epitope and the T cell epitope.

22. The method of numbered paragraph 21, wherein the heterologous spacer or linker is selected from the group consisting of Lys-, Gly-, Lys-Lys-Lys-, (α, ε-N)Lys, and ε-N-Lys-Lys-Lys-Lys, or a combination thereof.

23. The method of any one of numbered paragraphs 14 to 22, wherein the B cell epitope is located N-terminal to the T cell epitope.

24. The method of any one of numbered paragraphs 14 to 22, wherein the T cell epitope is located N-terminal to the B cell epitope.

25. The method of any one of numbered paragraphs 13 to 24, wherein the peptide immunogen construct is selected from a sequence of Table 3.

26. The method of any one of numbered paragraphs 13 to 25, wherein the peptide immunogen construct comprises:
(a) a B cell epitope comprising about 10 to about 25 amino acid residues from a C-terminal fragment of α-Syn corresponding to about amino acid G111 to about amino acid D135 of SEQ ID NO: 1;
(b) a T helper epitope comprising an amino acid sequence selected from the group consisting of SEQ ID NOs: 70-98; and
(c) an optional heterologous spacer selected from the group consisting of an amino acid, Lys-, Gly-, Lys-Lys-Lys-, (α, ε-N)Lys, and ε-N-Lys-Lys-Lys-Lys (SEQ ID NO: 148), or a combination thereof,
wherein the B cell epitope is covalently linked to the T helper epitope directly or through the optional heterologous spacer.

27. The method of numbered paragraph 26, wherein the B cell epitope is selected from the group consisting of SEQ ID NOs: 12-15, 17, and 49-63.

28. The method of numbered paragraph 26 or 27, wherein the T helper epitope is selected from the group consisting of SEQ ID NOs: 70, 71, 72, 73, 74, 75, 76, 77, 78, 79, 80, 81, 82, 83, 84, 85, 86, 87, 88, 89, 90, 91, 92, 93, 94, 95, 96, 97, and 98, for example, from any one of: SEQ ID NOs: 81, 83, and 84.

29. The method of any one of numbered paragraphs 26 to 28, wherein the optional heterologous spacer is (α, ε-N)Lys or ε-N-Lys-Lys-Lys-Lys (SEQ ID NO: 148).

30. The method of any one of numbered paragraphs 26 to 29, wherein the T helper epitope is covalently linked to the amino terminus of the B cell epitope.

31. The method of any one of numbered paragraphs 26 to 30, wherein the T helper epitope is covalently linked to the amino terminus of the B cell epitope through the optional heterologous spacer.

32. The method of any one of numbered paragraphs 26 to 31, wherein the peptide immunogen construct comprises the following formula:

(Th)ₘ-(A)ₙ-(α-Syn C-terminal fragment)-X

or

(α-Syn C-terminal fragment)-(A)ₙ-(Th)ₘ-X
wherein
Th is the T helper epitope;
A is the heterologous spacer;
(α-Syn C-terminal fragment) is the B cell epitope;
X is an α-COOH or α-CONH₂ of an amino acid;
m is from 1 to about 4; and
n is from 1 to about 10.

33. The method of any one of numbered paragraphs 26 to 32, wherein the peptide immunogen construct comprises an amino acid sequence selected from the group consisting of SEQ ID NOs: 107, 108, 111-113, and 115-147.

34. The method of any one of numbered paragraphs 13 to 33, wherein the peptide immunogen construct is in a stabilized immunostimulatory complex with a CpG oligodeoxynucleotide (ODN).

35. The method of any one of numbered paragraphs 1 to 34, wherein the immunotherapy is comprised within a composition, which optionally comprises more than one immunotherapy, e.g., more than one peptide immunogen construct.

36. The method of numbered paragraph 35, wherein the composition comprises peptide immunogen constructs comprising amino acid sequences of SEQ ID NOs: 112 and 113.

37. The method of numbered paragraph 35 or 36, wherein the composition is a pharmaceutical composition comprising the immunotherapy(ies) and a pharmaceutically acceptable delivery vehicle and/or adjuvant.

38. The method of numbered paragraph 37, wherein the composition comprises an adjuvant that comprises a mineral salt of aluminum, which optionally is selected from group consisting of Al(OH)₃ and AlPO₄.

39. The method of numbered paragraph 37 or 38, wherein:
(a) the peptide immunogen construct is selected from the group consisting of SEQ ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147, for example, the group consisting of SEQ ID NOs: 107, 108, 111-113, and 115-147; and
(b) the composition comprises an adjuvant that is a mineral salt of aluminum selected from the group consisting of Al(OH)₃ and AlPO₄.

40. The method of any one of numbered paragraphs 37 to 39, wherein:
(a) the peptide immunogen construct is selected from the group consisting of SEQ ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147, for example, the group consisting of SEQ ID NOs: 107, 108, 111-113, and 115-147; and
(b) the peptide immunogen construct is in the form of a stabilized immunostimulatory complex with a CpG ODN.

41. The method of any one of numbered paragraphs 1 to 12, wherein the immunotherapy comprises an antibody or an epitope-binding fragment thereof that specifically binds to the B cell epitope of a peptide immunogen construct of any one of numbered paragraphs 13 to 40, a B cell epitope of SEQ ID NO: 1 (e.g., the C-terminal region of SEQ ID NO: 1), or a peptide of Table 1 (e.g., any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69).

42. The method of any one of numbered paragraphs 1 to 41, comprising the use of two or more, three or more, four or more, or five or more immunotherapies.

43. The method of any one of numbered paragraphs 1 to 42, wherein the subject is diagnosed with rapid eye movement (REM) sleep behavior disorder (RBD).

44. The method of any one of numbered paragraphs 1 to 43, wherein the subject has one or more of hyposmia, REM sleep behavior disorder, excessive daytime sleepiness, depression, cognitive symptoms, autonomic nervous system dysfunction, olfactory loss, decreased color vision, slowing on quantitative motor testing, abnormal substantia nigra neuroimaging findings, or other prodromal symptom, e.g., as described herein.

45. The method of any one of numbered paragraphs 1 to 44, wherein the subject does not have any or any significant bradykinesia, rigidity, and/or tremor, or other symptom of a synucleinopathy that is not prodromal.

46. The method of any one of numbered paragraphs 1 to 45, wherein the immunotherapy comprises or consists of a peptide immunogen construct that comprises or consists of SEQ ID NO: 112.

47. A composition or kit for use in carrying out any one of the methods of any one of numbered paragraphs 1 to 46.

48. Use of a peptide immunogen construct or composition described herein in the preparation of a medicament for treating, preventing, inhibiting reducing, or slowing the development of one or more motor symptom of a synucleinopathy in a subject in need thereof,

Other embodiments are within the scope of the claims.

What is claimed is:

1. A method of preventing, reducing, inhibiting, or slowing the development of one or more motor symptom of a synucleinopathy in a subject in need thereof, the method comprising administering to the subject an effective amount of an immunotherapy targeting alpha-synuclein (α-syn).

2. The method of claim 1, wherein the one or more motor symptom of a synucleinopathy is selected from the group consisting of muscle rigidity, bradykinesia, tremor at rest, and postural instability.

3. A method of treating, preventing, reducing, or inhibiting one or more gastrointestinal symptom of a synucleinopathy in a subject in need thereof, the method comprising administering to the subject an effective amount of an immunotherapy targeting α-syn.

4. The method of claim 3, wherein the one or more gastrointestinal symptom is selected from the group consisting of: drooling, salivation, dysphagia, nausea, vomiting, dyspepsia, constipation, abdominal pain, gastroparesis, and fecal incontinence.

5. The method of claim 3, wherein the gastrointestinal symptom occurs in the colon of the subject.

6. A method of reducing the level of α-syn in the gastrointestinal tract (e.g., the colon) of a subject in need thereof, the method comprising administering to the subject an effective amount of an immunotherapy targeting α-syn.

7. The method of any one of claims 1 to 6, wherein the subject does not have one or more motor symptom of a synucleinopathy or exhibits only a minimal motor symptom of a synucleinopathy.

8. The method of claim 7, wherein the subject does not have one or more motor symptom of a synucleinopathy selected from the group consisting of muscle rigidity, bradykinesia, tremor at rest, and postural instability.

9. The method of any one of claims 1 to 6, wherein the subject has a synucleinopathy at an early, prodromal stage.

10. A method of inducing an immune response to α-syn in a subject, inhibiting α-syn aggregation in a subject, or reducing the amount of α-syn aggregates in a subject, the method comprising administering an effective amount of an immunotherapy targeting α-syn to the subject, wherein the subject has a synucleinopathy at an early, prodromal stage.

11. The method of any one of claims 1 to 6 or 10, wherein the synucleinopathy is selected from the group consisting of Parkinson's disease (PD), Parkinson's disease dementia (PDD), dementia with Lewy bodies (DLB), multiple system atrophy (MSA), neuroaxonal dystrophies, and pure autonomic failure (PAF).

12. The method of any one of claims 1 to 6 or 10, wherein the immunotherapy comprises a peptide, a protein (e.g., an antibody), a fragment or fusion of a peptide or a protein (e.g., an antibody), or a nucleic acid molecule (e.g., an mRNA or a nucleic acid in a vector) encoding one of said molecules.

13. The method of any one of claims 1 to 6 or 10, wherein the immunotherapy comprises a peptide immunogen construct.

14. The method of claim 13, wherein the peptide immunogen construct comprises a B cell epitope, a heterologous T cell epitope, and an optional linker.

15. The method of claim 14, wherein the B cell epitope induces an immune response against α-syn.

16. The method of claim 15, wherein the B cell epitope comprises a peptide of the C-terminal region of an α-syn protein, wherein the peptide optionally is about 10 to about 25 amino acids in length.

17. The method of claim 16, wherein the α-syn protein comprises the sequence of SEQ ID NO: 1.

18. The method of claim 14, wherein the B cell epitope comprises a peptide selected from a sequence of Table 1 (e.g., any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69).

19. The method of claim 14, wherein the heterologous T cell epitope is derived from a pathogenic protein.

20. The method of claim 14, wherein the heterologous T cell epitope comprises a sequence selected from a sequence of Table 2.

21. The method of claim 14, wherein the peptide comprises a heterologous spacer or linker between the B cell epitope and the T cell epitope.

22. The method of claim 21, wherein the heterologous spacer or linker is selected from the group consisting of Lys-, Gly-, Lys-Lys-Lys-, (α, ε-N)Lys, and ε-N-Lys-Lys-Lys-Lys, or a combination thereof.

23. The method of claim 14, wherein the B cell epitope is located N-terminal to the T cell epitope.

24. The method of claim 14, wherein the T cell epitope is located N-terminal to the B cell epitope.

25. The method of claim 13, wherein the peptide immunogen construct is selected from a sequence of Table 3.

26. The method of claim 13, wherein the peptide immunogen construct comprises:
(a) a B cell epitope comprising about 10 to about 25 amino acid residues from a C-terminal fragment of α-Syn corresponding to about amino acid G111 to about amino acid D135 of SEQ ID NO: 1;
(b) a T helper epitope comprising an amino acid sequence selected from the group consisting of SEQ ID NOs: 70-98; and
(c) an optional heterologous spacer selected from the group consisting of an amino acid, Lys-, Gly-, Lys-Lys-Lys-, (α, ε-N)Lys, and ε-N-Lys-Lys-Lys-Lys (SEQ ID NO: 148), or a combination thereof,
wherein the B cell epitope is covalently linked to the T helper epitope directly or through the optional heterologous spacer.

27. The method of claim 26, wherein the B cell epitope is selected from the group consisting of SEQ ID NOs: 12-15, 17, and 49-63.

28. The method of claim 26, wherein the T helper epitope is selected from the group consisting of SEQ ID NOs: 70, 71, 72, 73, 74, 75, 76, 77, 78, 79, 80, 81, 82, 83, 84, 85, 86, 87, 88, 89, 90, 91, 92, 93, 94, 95, 96, 97, and 98, for example, from any one of: SEQ ID NOs: 81, 83, and 84.

29. The method of claim 26, wherein the optional heterologous spacer is (α, ε-N)Lys or ε-N-Lys-Lys-Lys-Lys (SEQ ID NO: 148).

30. The method of claim 26, wherein the T helper epitope is covalently linked to the amino terminus of the B cell epitope.

31. The method of claim 26, wherein the T helper epitope is covalently linked to the amino terminus of the B cell epitope through the optional heterologous spacer.

32. The method of claim 26, wherein the peptide immunogen construct comprises the following formula:
(Th)ₘ-(A)ₙ-(α-Syn C-terminal fragment)-X
or
(α-Syn C-terminal fragment)-(A)ₙ-(Th)ₘ-X
wherein
Th is the T helper epitope;
A is the heterologous spacer;
(α-Syn C-terminal fragment) is the B cell epitope;
X is an α-COOH or α-CONH2 of an amino acid;
m is from 1 to about 4; and
n is from 1 to about 10.

33. The method of claim 26, wherein the peptide immunogen construct comprises an amino acid sequence selected from the group consisting of SEQ ID NOs: 107, 108, 111-113, and 115-147.

34. The method of claim 13, wherein the peptide immunogen construct is in a stabilized immunostimulatory complex with a CpG oligodeoxynucleotide (ODN).

35. The method of claim 1 to 6 or 10, wherein the immunotherapy is comprised within a composition, which optionally comprises more than one immunotherapy, e.g., more than one peptide immunogen construct.

36. The method of claim 35, wherein the composition comprises peptide immunogen constructs comprising amino acid sequences of SEQ ID NOs: 112 and 113.

37. The method of claim 35, wherein the composition is a pharmaceutical composition comprising the immunotherapy(ies) and a pharmaceutically acceptable delivery vehicle and/or adjuvant.

38. The method of claim 37, wherein the composition comprises an adjuvant that comprises a mineral salt of aluminum, which optionally is selected from group consisting of Al(OH)₃ and AlPO₄.

39. The method of claim 37, wherein:
(a) the peptide immunogen construct is selected from the group consisting of SEO ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147, for example, the group consisting of SEQ ID NOs: 107, 108, 111-113, and 115-147; and
(b) the composition comprises an adjuvant that is a mineral salt of aluminum selected from the group consisting of Al(OH)₃ and AlPO₄.

40. The method of claim 37, wherein:
(a) the peptide immunogen construct is selected from the group consisting of SEO ID NOs: 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, and 147, for example, the group consisting of SEQ ID NOs: 107, 108, 111-113, and 115-147; and
(b) the peptide immunogen construct is in the form of a stabilized immunostimulatory complex with a CpG ODN.

41. The method of claim 1, wherein the immunotherapy comprises an antibody or an epitope-binding fragment thereof that specifically binds to the B cell epitope of a peptide immunogen construct of any one of claims 13 to 40, a B cell epitope of SEQ ID NO: 1 (e.g., the C-terminal region of SEQ ID NO: 1), or a peptide of Table 1 (e.g., any one of SEQ ID NOs: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, and 69).

42. The method of any one of claims 1 to 6 or 10, comprising the use of two or more, three or more, four or more, or five or more immunotherapies.

43. The method of any one of claims 1 to 6 or 10, wherein the subject is diagnosed with rapid eye movement (REM) sleep behavior disorder (RBD).

44. The method of any one of claims 1 to 6 or 10, wherein the subject has one or more of hyposmia, REM sleep behavior disorder, excessive daytime sleepiness, depression, cognitive symptoms, autonomic nervous system dysfunction, olfactory loss, decreased color vision, slowing on quantitative motor testing, abnormal substantia nigra neuroimaging findings, or other prodromal symptom, e.g., as described herein.

45. The method of any one of claims 1 to 6 or 10, wherein the subject does not have any or any significant bradykinesia, rigidity, and/or tremor, or other symptom of a synucleinopathy that is not prodromal.

46. The method of any one of claims 1 to 6 or 10, wherein the immunotherapy comprises or consists of a peptide immunogen construct that comprises or consists of SEQ ID NO: 112.

47. A composition or kit for use in carrying out any one of the methods of any one of claims 1 to 6 or 10.

48. Use of a peptide immunogen construct or composition described herein in the preparation of a medicament for treating, preventing, inhibiting reducing, or slowing the development of one or more motor symptom of a synucleinopathy in a subject in need thereof.


SEQUENCE LISTING (149 sequences)

SEQ ID NO: 1 - protein, 140 aa, Homo sapiens
  PEPTIDE 1..140: note = alpha-Synuclein 1-140
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - skipped

SEQ ID NO: 3 - protein, 61 aa, Homo sapiens
  PEPTIDE 1..61: note = alpha-Synuclein 80-140
KTVEGAGSIAAATGFVKKDQLGKNEEGAPQEGILEDMPVDPDNEAYEMPSEEGYQDYEPEA

SEQ ID NO: 4 - protein, 56 aa, Homo sapiens
  PEPTIDE 1..56: note = alpha-Synuclein 85-140
AGSIAAATGFVKKDQLGKNEEGAPQEGILEDMPVDPDNEAYEMPSEEGYQDYEPEA

SEQ ID NO: 5 - protein, 50 aa, Homo sapiens
  PEPTIDE 1..50: note = alpha-Synuclein 91-140
ATGFVKKDQLGKNEEGAPQEGILEDMPVDPDNEAYEMPSEEGYQDYEPEA

SEQ ID NO: 6 - protein, 40 aa, Homo sapiens
  PEPTIDE 1..40: note = alpha-Synuclein 101-140
GKNEEGAPQEGILEDMPVDPDNEAYEMPSEEGYQDYEPEA

SEQ ID NO: 7 - protein, 30 aa, Homo sapiens
  PEPTIDE 1..30: note = alpha-Synuclein 111-140
GILEDMPVDPDNEAYEMPSEEGYQDYEPEA

SEQ ID NO: 8 - protein, 20 aa, Homo sapiens
  PEPTIDE 1..20: note = alpha-Synuclein 121-140
DNEAYEMPSEEGYQDYEPEA

SEQ ID NO: 9 - protein, 15 aa, Homo sapiens
EMPSEEGYQDYEPEA

SEQ ID NO: 10 - protein, 39 aa, Homo sapiens
  PEPTIDE 1..39: note = alpha-Synuclein 97-135
KDQLGKNEEGAPQEGILEDMPVDPDNEAYEMPSEEGYQD

SEQ ID NO: 11 - protein, 35 aa, Homo sapiens
  PEPTIDE 1..35: note = alpha-Synuclein 101-135
GKNEEGAPQEGILEDMPVDPDNEAYEMPSEEGYQD

SEQ ID NO: 12 - protein, 25 aa, Homo sapiens
  PEPTIDE 1..25: note = alpha-Synuclein 111-135
GILEDMPVDPDNEAYEMPSEEGYQD

SEQ ID NO: 13 - protein, 15 aa, Homo sapiens
  PEPTIDE 1..15: note = alpha-Synuclein 121-135
DNEAYEMPSEEGYQD

SEQ ID NO: 14 - protein, 13 aa, Homo sapiens
  PEPTIDE 1..13: note = alpha-Synuclein 123-135
EAYEMPSEEGYQD

SEQ ID NO: 15 - protein, 10 aa, Homo sapiens
  PEPTIDE 1..10: note = alpha-Synuclein 126-135
EMPSEEGYQD

SEQ ID NO: 16 - protein, 32 aa, Homo sapiens
  PEPTIDE 1..32: note = alpha-Synuclein 101-132
GKNEEGAPQEGILEDMPVDPDNEAYEMPSEEG

SEQ ID NO: 17 - protein, 22 aa, Homo sapiens
  PEPTIDE 1..22: note = alpha-Synuclein 111-132
GILEDMPVDPDNEAYEMPSEEG

SEQ ID NO: 18 - protein, 10 aa, Homo sapiens
  PEPTIDE 1..10: note = alpha-Synuclein 80-89
KTVEGAGSIA

SEQ ID NO: 19 - protein, 10 aa, Homo sapiens
  PEPTIDE 1..10: note = alpha-Synuclein 81-90
TVEGAGSIAA

SEQ ID NO: 20 - protein, 10 aa, Homo sapiens
  PEPTIDE 1..10: note = alpha-Synuclein 82-91
VEGAGSIAAA

SEQ ID NO: 21 - protein, 10 aa, Homo sapiens
  PEPTIDE 1..10: note = alpha-Synuclein 83-92
EGAGSIAAAT

SEQ ID NO: 22 - protein, 10 aa, Homo sapiens
  PEPTIDE 1..10: note = alpha-Synuclein 84-93
GAGSIAAATG

SEQ ID NO: 23 - protein, 10 aa, Homo sapiens
  PEPTIDE 1..10: note = alpha-Synuclein 85-94
AGSIAAATGF

SEQ ID NO: 24 - protein, 10 aa, Homo sapiens
  PEPTIDE 1..10: note = alpha-Synuclein 86-95
GSIAAATGFV

SEQ ID NO: 25 - protein, 10 aa, Homo sapiens
  PEPTIDE 1..10: note = alpha-Synuclein 87-96
SIAAATGFVK

SEQ ID NO: 26 - protein, 10 aa, Homo sapiens
  PEPTIDE 1..10: note = alpha-Synuclein 88-97
IAAATGFVKK

SEQ ID NO: 27 - protein, 10 aa, Homo sapiens
  PEPTIDE 1..10: note = alpha-Synuclein 89-98
AAATGFVKKD

SEQ ID NO: 28 - protein, 10 aa, Homo sapiens
  PEPTIDE 1..10: note = alpha-Synuclein 90-99
AATGFVKKDQ

SEQ ID NO: 29 - protein, 10 aa, Homo sapiens
  PEPTIDE 1..10: note = alpha-Synuclein 91-100
ATGFVKKDQL

SEQ ID NO: 30 - protein, 10 aa, Homo sapiens
  PEPTIDE 1..10: note = alpha-Synuclein 92-101
TGFVKKDQLG

SEQ ID NO: 31 - protein, 10 aa, Homo sapiens
  PEPTIDE 1..10: note = alpha-Synuclein 93-102
GFVKKDQLGK

SEQ ID NO: 32 - protein, 10 aa, Homo sapiens
  PEPTIDE 1..10: note = alpha-Synuclein 94-103
FVKKDQLGKN

SEQ ID NO: 33 - protein, 10 aa, Homo sapiens
  PEPTIDE 1..10: note = alpha-Synuclein 95-104
VKKDQLGKNE

SEQ ID NO: 34 - protein, 10 aa, Homo sapiens
  PEPTIDE 1..10: note = alpha-Synuclein 96-105
KKDQLGKNEE

SEQ ID NO: 35 - protein, 10 aa, Homo sapiens
  PEPTIDE 1..10: note = alpha-Synuclein 97-106
KDQLGKNEEG

SEQ ID NO: 36 - protein, 10 aa, Homo sapiens
  PEPTIDE 1..10: note = alpha-Synuclein 98-107
DQLGKNEEGA

SEQ ID NO: 37 - protein, 10 aa, Homo sapiens
  PEPTIDE 1..10: note = alpha-Synuclein 99-108
QLGKNEEGAP

SEQ ID NO: 38 - protein, 10 aa, Homo sapiens
  PEPTIDE 1..10: note = alpha-Synuclein 100-109
LGKNEEGAPQ

SEQ ID NO: 39 - protein, 10 aa, Homo sapiens
  PEPTIDE 1..10: note = alpha-Synuclein 101-110
GKNEEGAPQE

SEQ ID NO: 40 - protein, 10 aa, Homo sapiens
  PEPTIDE 1..10: note = alpha-Synuclein 102-111
KNEEGAPQEG

SEQ ID NO: 41 - protein, 10 aa, Homo sapiens
  PEPTIDE 1..10: note = alpha-Synuclein 103-112
NEEGAPQEGI

SEQ ID NO: 42 - protein, 10 aa, Homo sapiens
  PEPTIDE 1..10: note = alpha-Synuclein 104-113
EEGAPQEGIL

SEQ ID NO: 43 - protein, 10 aa, Homo sapiens
  PEPTIDE 1..10: note = alpha-Synuclein 105-114
EGAPQEGILE

SEQ ID NO: 44 - protein, 10 aa, Homo sapiens
  PEPTIDE 1..10: note = alpha-Synuclein 106-115
GAPQEGILED

SEQ ID NO: 45 - protein, 10 aa, Homo sapiens
  PEPTIDE 1..10: note = alpha-Synuclein 107-116
APQEGILEDM

SEQ ID NO: 46 - protein, 10 aa, Homo sapiens
  PEPTIDE 1..10: note = alpha-Synuclein 108-117
PQEGILEDMP

SEQ ID NO: 47 - protein, 10 aa, Homo sapiens
  PEPTIDE 1..10: note = alpha-Synuclein 109-118
QEGILEDMPV

SEQ ID NO: 48 - protein, 10 aa, Homo sapiens
  PEPTIDE 1..10: note = alpha-Synuclein 110-119
EGILEDMPVD

SEQ ID NO: 49 - protein, 10 aa, Homo sapiens
  PEPTIDE 1..10: note = alpha-Synuclein 111-120
GILEDMPVDP

SEQ ID NO: 50 - protein, 10 aa, Homo sapiens
  PEPTIDE 1..10: note = alpha-Synuclein 112-121
ILEDMPVDPD

SEQ ID NO: 51 - protein, 10 aa, Homo sapiens
  PEPTIDE 1..10: note = alpha1-Synuclein 113-122
LEDMPVDPDN

SEQ ID NO: 52 - protein, 10 aa, Homo sapiens
  PEPTIDE 1..10: note = alpha-Synuclein 114-123
EDMPVDPDNE

SEQ ID NO: 53 - protein, 10 aa, Homo sapiens
  PEPTIDE 1..10: note = alpha-Synuclein 115-124
DMPVDPDNEA

SEQ ID NO: 54 - protein, 10 aa, Homo sapiens
  PEPTIDE 1..10: note = alpha-Synuclein 116-125
MPVDPDNEAY

SEQ ID NO: 55 - protein, 10 aa, Homo sapiens
  PEPTIDE 1..10: note = alpha-Synuclein 117-126
PVDPDNEAYE

SEQ ID NO: 56 - protein, 10 aa, Homo sapiens
  PEPTIDE 1..10: note = alpha-Synuclein 118-127
VDPDNEAYEM

SEQ ID NO: 57 - protein, 10 aa, Homo sapiens
  PEPTIDE 1..10: note = alpha-Synuclein 119-128
DPDNEAYEMP

SEQ ID NO: 58 - protein, 10 aa, Homo sapiens
  PEPTIDE 1..10: note = alpha-Synuclein 120-129
PDNEAYEMPS

SEQ ID NO: 59 - protein, 10 aa, Homo sapiens
  PEPTIDE 1..10: note = alpha-Synuclein 121-130
DNEAYEMPSE

SEQ ID NO: 60 - protein, 10 aa, Homo sapiens
  PEPTIDE 1..10: note = alpha-Synuclein 122-131
NEAYEMPSEE

SEQ ID NO: 61 - protein, 10 aa, Homo sapiens
  PEPTIDE 1..10: note = alpha-Synuclein 123-132
EAYEMPSEEG

SEQ ID NO: 62 - protein, 10 aa, Homo sapiens
  PEPTIDE 1..10: note = alpha-Synuclein 124-133
AYEMPSEEGY

SEQ ID NO: 63 - protein, 10 aa, Homo sapiens
  PEPTIDE 1..10: note = alpha-Synuclein 125-134
YEMPSEEGYQ

SEQ ID NO: 64 - protein, 10 aa, Homo sapiens
  PEPTIDE 1..10: note = alpha-Synuclein 126-135
EMPSEEGYQD

SEQ ID NO: 65 - protein, 10 aa, Homo sapiens
  PEPTIDE 1..10: note = alpha-Synuclein 127-136
MPSEEGYQDY

SEQ ID NO: 66 - protein, 10 aa, Homo sapiens
  PEPTIDE 1..10: note = alpha-Synuclein 128-137
PSEEGYQDYE

SEQ ID NO: 67 - protein, 10 aa, Homo sapiens
  PEPTIDE 1..10: note = alpha-Synuclein 129-138
SEEGYQDYEP

SEQ ID NO: 68 - protein, 10 aa, Homo sapiens
  PEPTIDE 1..10: note = alpha-Synuclein 130-139
EEGYQDYEPE

SEQ ID NO: 69 - protein, 10 aa, Homo sapiens
  PEPTIDE 1..10: note = alpha-Synuclein 131-140
EGYQDYEPEA

SEQ ID NO: 70 - protein, 17 aa, Clostridium tetani
  PEPTIDE 1..17: note = Clostridium tetani 1 Th
KKQYIKANSKFIGITEL

SEQ ID NO: 71 - protein, 15 aa, Measles morbillivirus
  PEPTIDE 1..15: note = MvF1 Th
LSEIKGVIVHRLEGV

SEQ ID NO: 72 - protein, 24 aa, Bordetella pertussis
  PEPTIDE 1..24: note = Bordetella pertussis Th
GAYARCPNGTRALTVAELRGNAEL

SEQ ID NO: 73 - protein, 17 aa, Clostridium tetani
  PEPTIDE 1..17: note = Clostridium tetani 2 Th
WVRDIIDDFTNESSQKT

SEQ ID NO: 74 - protein, 23 aa, unidentified
  PEPTIDE 1..23: note = Diphtheria Th
DSETADNLEKTVAALSILPGHGC

SEQ ID NO: 75 - protein, 21 aa, unidentified
  PEPTIDE 1..21: note = Plasmodium falciparum Th
DHEKKHAKMEKASSVFNVVNS

SEQ ID NO: 76 - protein, 17 aa, Schistosoma mansoni
  PEPTIDE 1..17: note = Schistosoma mansoni Th
KWFKTNAPNGVDEKHRH

SEQ ID NO: 77 - protein, 25 aa, unidentified
  PEPTIDE 1..25: note = Cholera Toxin Th
ALNIWDRFDVFCTLGATTGYLKGNS

SEQ ID NO: 78 - protein, 15 aa, Measles morbillivirus
  PEPTIDE 1..15: note = MvF 2 Th
ISEIKGVIVHKIEGI

SEQ ID NO: 79 - protein, 22 aa, Measles morbillivirus
  VARIANT 7: note = serine or threonine
  VARIANT 10: note = lysine or arginine
  VARIANT 11: note = glycine or threonine
  VARIANT 15: note = histidine or threonine
  VARIANT 16: note = lysine or arginine
  VARIANT 19: note = glycine or threonine
  PEPTIDE 1..22: note = KKKMvF 3 Th
KKKISIXEIXXVIVXXIEXILF

SEQ ID NO: 80 - protein, 18 aa, Hepatitis B virus
  VARIANT 1: note = lysine or arginine
  VARIANT 2: note = lysine or arginine
  VARIANT 3: note = lysine or arginine
  VARIANT 4: note = leucine, isoleucine, valine or phenylalanine
  VARIANT 5: note = phenylalanine, lysine or arginine
  VARIANT 6: note = leucine, isoleucine, valine or phenylalanine
  VARIANT 7: note = leucine, isoleucine, valine or phenylalanine
  VARIANT 9: note = lysine or arginine
  VARIANT 10: note = leucine, isoleucine, valine or phenylalanine
  VARIANT 11: note = leucine, isoleucine, valine or phenylalanine
  VARIANT 13: note = leucine, isoleucine, valine or phenylalanine
  VARIANT 15: note = glutamine, leucine, isoleucine, valine or phenylalanine
  VARIANT 17: note = leucine, isoleucine, valine or phenylalanine
  VARIANT 18: note = aspartic acid or arginine
  PEPTIDE 1..18: note = HBsAg 1 Th
XXXXXXXTXXXTXPXSXX

SEQ ID NO: 81 - protein, 19 aa, Measles morbillivirus
  VARIANT 4: note = serine or threonine
  VARIANT 7: note = lysine or arginine
  VARIANT 8: note = glycine or threonine
  VARIANT 12: note = histidine or threonine
  VARIANT 13: note = lysine or arginine
  PEPTIDE 1..19: note = MvF 4 Th
ISIXEIXXVIVXXIETILF

SEQ ID NO: 82 - protein, 18 aa, Hepatitis B virus
  VARIANT 4: note = isoleucine or phenylalanine
  VARIANT 5: note = isoleucine or phenylalanine
  VARIANT 6: note = threonine or leucine
  VARIANT 7: note = isoleucine or leucine
  VARIANT 11: note = isoleucine or leucine
  VARIANT 14: note = proline or isoleucine
  VARIANT 15: note = glutamine or threonine
  VARIANT 16: note = serine or threonine
  VARIANT 17: note = leucine or isoleucine
  PEPTIDE 1..18: note = HBsAg 2 Th
KKKXXXXTRIXTIXXXXD

SEQ ID NO: 83 - protein, 19 aa, Measles morbillivirus
  PEPTIDE 1..19: note = MvF 5 Th
ISITEIKGVIVHRIETILF

SEQ ID NO: 84 - protein, 18 aa, Hepatitis B virus
  PEPTIDE 1..18: note = HBsAg 3 Th
KKKIITITRIITIITTID

SEQ ID NO: 85 - protein, 11 aa, unidentified
  PEPTIDE 1..11: note = Influenza Matrix protein 1_1 Th
FVFTLTVPSER

SEQ ID NO: 86 - protein, 15 aa, unidentified
  PEPTIDE 1..15: note = Influenza Matrix protein 1_2 Th
SGPLKAEIAQRLEDV

SEQ ID NO: 87 - protein, 9 aa, unidentified
  PEPTIDE 1..9: note = Influenza Non-structural protein 1 Th
DRLRRDQKS

SEQ ID NO: 88 - protein, 19 aa, Human gammaherpesvirus 4
  PEPTIDE 1..19: note = EBV BHRF1 Th
AGLTLSLLVICSYLFISRG

SEQ ID NO: 89 - protein, 15 aa, Clostridium tetani
  PEPTIDE 1..15: note = Clostridium tetani TT1 Th
QYIKANSKFIGITEL

SEQ ID NO: 90 - protein, 20 aa, Human gammaherpesvirus 4
  PEPTIDE 1..20: note = EBV EBNA-1 Th
PGPLRESIVCYFMVFLQTHI

SEQ ID NO: 91 - protein, 21 aa, Clostridium tetani
  PEPTIDE 1..21: note = Clostridium tetani TT2 Th
FNNFTVSFWLRVPKVSASHLE

SEQ ID NO: 92 - protein, 16 aa, Clostridium tetani
  PEPTIDE 1..16: note = Clostridium tetani TT3 Th
KFIIKRYTPNNEIDSF

SEQ ID NO: 93 - protein, 16 aa, Clostridium tetani
  PEPTIDE 1..16: note = Clostridium tetani TT4 Th
VSIDKFRIFCKALNPK

SEQ ID NO: 94 - protein, 18 aa, Human gammaherpesvirus 4
  PEPTIDE 1..18: note = EBV CP Th
VPGLYSPCRAFFNKEELL

SEQ ID NO: 95 - protein, 14 aa, Human betaherpesvirus 5
  PEPTIDE 1..14: note = HCMV IE1 Th
DKREMWMACIKELH

SEQ ID NO: 96 - protein, 15 aa, Human gammaherpesvirus 4
  PEPTIDE 1..15: note = EBV GP340 Th
TGHGARTSTEPTTDY

SEQ ID NO: 97 - protein, 13 aa, Human gammaherpesvirus 4
  PEPTIDE 1..13: note = EBV BPLF1 Th
KELKRQYEKKLRQ

SEQ ID NO: 98 - protein, 11 aa, Human gammaherpesvirus 4
  PEPTIDE 1..11: note = EBV EBNA-2 Th
TVFYNIPPMPL

SEQ ID NO: 99 - protein, 38 aa, synthetic construct
  VARIANT 4: note = serine or threonine
  VARIANT 7: note = lysine or arginine
  VARIANT 8: note = glycine or threonine
  VARIANT 12: note = histidine or threonine
  VARIANT 13: note = lysine or arginine
  PEPTIDE 1..19: note = MvF 4 Th
  SITE 20: note = epsilon-lysine
  PEPTIDE 20..23: note = epsilon lysine - lysine-lysine-lysine as a spacer
  PEPTIDE 24..38: note = alpha-Synuclein 126-140
ISIXEIXXVIVXXIETILFKKKKEMPSEEGYQDYEPEA

SEQ ID NO: 100 - protein, 43 aa, synthetic construct
  VARIANT 4: note = serine or threonine
  VARIANT 7: note = lysine or arginine
  VARIANT 8: note = glycine or threonine
  VARIANT 12: note = histidine or threonine
  VARIANT 13: note = lysine or arginine
  PEPTIDE 1..19: note = MvF 4 Th
  SITE 20: note = epsilon-lysine
  PEPTIDE 20..23: note = epsilon lysine - lysine-lysine-lysine as a spacer
  PEPTIDE 24..43: note = alpha-Synuclein 121-140
ISIXEIXXVIVXXIETILFKKKKDNEAYEMPSEEGYQDYEPEA

SEQ ID NO: 101 - protein, 53 aa, synthetic construct
  VARIANT 4: note = serine or threonine
  VARIANT 7: note = lysine or arginine
  VARIANT 8: note = glycine or threonine
  VARIANT 12: note = histidine or threonine
  VARIANT 13: note = lysine or arginine
  PEPTIDE 1..19: note = MvF 4 Th
  SITE 20: note = epsilon-lysine
  PEPTIDE 20..23: note = epsilon lysine - lysine-lysine-lysine as a spacer
  PEPTIDE 24..53: note = alpha-Synuclein 111-140
ISIXEIXXVIVXXIETILFKKKKGILEDMPVDPDNEAYEMPSEEGYQDYEPEA

SEQ ID NO: 102 - protein, 63 aa, synthetic construct
  VARIANT 4: note = serine or threonine
  VARIANT 7: note = lysine or arginine
  VARIANT 8: note = glycine or threonine
  VARIANT 12: note = histidine or threonine
  VARIANT 13: note = lysine or arginine
  PEPTIDE 1..19: note = MvF 4 Th
  SITE 20: note = epsilon-lysine
  PEPTIDE 20..23: note = epsilon lysine - lysine-lysine-lysine as a spacer
  PEPTIDE 24..63: note = alpha-Synuclein 126-140
ISIXEIXXVIVXXIETILFKKKKGKNEEGAPQEGILEDMPVDPDNEAYEMPSEEGYQDYEPEA

SEQ ID NO: 103 - protein, 63 aa, synthetic construct
  PEPTIDE 1..19: note = MvF5 Th
  SITE 20: note = epsilon-lysine
  PEPTIDE 20..23: note = epsilon lysine - lysine-lysine-lysine as a spacer
  PEPTIDE 24..63: note = alpha-Synuclein 101-140
ISITEIKGVIVHRIETILFKKKKGKNEEGAPQEGILEDMPVDPDNEAYEMPSEEGYQDYEPEA

SEQ ID NO: 104 - protein, 62 aa, synthetic construct
  PEPTIDE 1..18: note = HBsAg3 Th
  SITE 19: note = epsilon-lysine
  PEPTIDE 19..22: note = epsilon lysine - lysine-lysine-lysine as a spacer
  PEPTIDE 23..62: note = alpha-Synuclein 101-140
KKKIITITRIITIITTIDKKKKGKNEEGAPQEGILEDMPVDPDNEAYEMPSEEGYQDYEPEA

SEQ ID NO: 105 - protein, 73 aa, synthetic construct
  VARIANT 4: note = serine or threonine
  VARIANT 7: note = lysine or arginine
  VARIANT 8: note = glycine or threonine
  VARIANT 12: note = histidine or threonine
  VARIANT 13: note = lysine or arginine
  PEPTIDE 1..19: note = MvF 4 Th
  SITE 20: note = epsilon-lysine
  PEPTIDE 20..23: note = epsilon lysine - lysine-lysine-lysine as a spacer
  PEPTIDE 24..73: note = alpha-Synuclein 91-140
ISIXEIXXVIVXXIETILFKKKKATGFVKKDQLGKNEEGAPQEGILEDMPVDPDNEAYEMPSEEGYQDYEPEA

SEQ ID NO: 106 - protein, 79 aa, synthetic construct
  VARIANT 4: note = serine or threonine
  VARIANT 7: note = lysine or arginine
  VARIANT 8: note = glycine or threonine
  VARIANT 12: note = histidine or threonine
  VARIANT 13: note = lysine or arginine
  PEPTIDE 1..19: note = MvF 4 Th
  SITE 20: note = epsilon-lysine
  PEPTIDE 20..23: note = epsilon lysine - lysine-lysine-lysine as a spacer
  PEPTIDE 24..79: note = alpha-Synuclein 85-140
ISIXEIXXVIVXXIETILFKKKKAGSIAAATGFVKKDQLGKNEEGAPQEGILEDMPVDPDNEAYEMPSEEGYQDYEPEA

SEQ ID NO: 107 - protein, 38 aa, synthetic construct
  PEPTIDE 1..19: note = MvF5 Th
  SITE 20: note = epsilon-lysine
  PEPTIDE 20..23: note = epsilon lysine - lysine-lysine-lysine as a spacer
  PEPTIDE 24..38: note = alpha-Synuclein 121-135
ISITEIKGVIVHRIETILFKKKKDNEAYEMPSEEGYQD

SEQ ID NO: 108 - protein, 48 aa, synthetic construct
  PEPTIDE 1..19: note = MvF5 Th
  SITE 20: note = epsilon-lysine
  PEPTIDE 20..23: note = epsilon lysine - lysine-lysine-lysine as a spacer
  PEPTIDE 24..48: note = alpha-Synuclein 111-135
ISITEIKGVIVHRIETILFKKKKGILEDMPVDPDNEAYEMPSEEGYQD

SEQ ID NO: 109 - protein, 58 aa, synthetic construct
  PEPTIDE 1..19: note = MvF5 Th
  SITE 20: note = epsilon-lysine
  PEPTIDE 20..23: note = epsilon lysine - lysine-lysine-lysine as a spacer
  PEPTIDE 24..58: note = alpha-Synuclein 101-135
ISITEIKGVIVHRIETILFKKKKGKNEEGAPQEGILEDMPVDPDNEAYEMPSEEGYQD

SEQ ID NO: 110 - protein, 62 aa, synthetic construct
  PEPTIDE 1..19: note = MvF5 Th
  SITE 20: note = epsilon-lysine
  PEPTIDE 20..23: note = epsilon lysine - lysine-lysine-lysine as a spacer
  PEPTIDE 24..62: note = alpha-Synuclein 97-135
ISITEIKGVIVHRIETILFKKKKKDQLGKNEEGAPQEGILEDMPVDPDNEAYEMPSEEGYQD

SEQ ID NO: 111 - protein, 36 aa, synthetic construct
  PEPTIDE 1..19: note = MvF5 Th
  SITE 20: note = epsilon-lysine
  PEPTIDE 20..23: note = epsilon lysine - lysine-lysine-lysine as a spacer
  PEPTIDE 24..36: note = alpha-Synuclein 123-135
ISITEIKGVIVHRIETILFKKKKEAYEMPSEEGYQD

SEQ ID NO: 112 - protein, 33 aa, synthetic construct
  PEPTIDE 1..19: note = MvF5 Th
  SITE 20: note = epsilon-lysine
  PEPTIDE 20..23: note = epsilon lysine - lysine-lysine-lysine as a spacer
  PEPTIDE 24..33: note = alpha-Synuclein 126-135
ISITEIKGVIVHRIETILFKKKKEMPSEEGYQD

SEQ ID NO: 113 - protein, 45 aa, synthetic construct
  PEPTIDE 1..19: note = MvF5 Th
  SITE 20: note = epsilon-lysine
  PEPTIDE 20..23: note = epsilon lysine - lysine-lysine-lysine as a spacer
  PEPTIDE 24..45: note = alpha-Synuclein 111-132
ISITEIKGVIVHRIETILFKKKKGILEDMPVDPDNEAYEMPSEEG

SEQ ID NO: 114 - protein, 55 aa, synthetic construct
  PEPTIDE 1..19: note = MvF5 Th
  SITE 20: note = epsilon-lysine
  PEPTIDE 20..23: note = epsilon lysine - lysine-lysine-lysine as a spacer
  PEPTIDE 24..55: note = alpha-Synuclein 101-132
ISITEIKGVIVHRIETILFKKKKGKNEEGAPQEGILEDMPVDPDNEAYEMPSEEG

SEQ ID NO: 115 - protein, 45 aa, synthetic construct
  PEPTIDE 1..19: note = MvF 5 Th
  SITE 20: note = epsilon-lysine
  PEPTIDE 20..23: note = epsilon lysine - lysine-lysine-lysine as a spacer
  PEPTIDE 24..45: note = Mouse alpha-Synuclein 111-132
ISISEIKGVIVHKIETILFKKKKGILEDMPVDPGSEAYEMPSEEG

SEQ ID NO: 116 - protein, 33 aa, synthetic construct
  VARIANT 4: note = serine or threonine
  VARIANT 7: note = lysine or arginine
  VARIANT 8: note = glycine or threonine
  VARIANT 12: note = histidine or threonine
  VARIANT 13: note = lysine or arginine
  PEPTIDE 1..19: note = MvF 4 Th
  SITE 20: note = epsilon-lysine
  PEPTIDE 20..23: note = epsilon lysine - lysine-lysine-lysine as a spacer
  PEPTIDE 24..33: note = alpha-Synuclein 126-135
ISIXEIXXVIVXXIETILFKKKKEMPSEEGYQD

SEQ ID NO: 117 - protein, 45 aa, synthetic construct
  VARIANT 4: note = serine or threonine
  VARIANT 7: note = lysine or arginine
  VARIANT 8: note = glycine or threonine
  VARIANT 12: note = histidine or threonine
  VARIANT 13: note = lysine or arginine
  PEPTIDE 1..19: note = MvF 4 Th
  SITE 20: note = epsilon-lysine
  PEPTIDE 20..23: note = epsilon lysine - lysine-lysine-lysine as a spacer
  PEPTIDE 24..45: note = alpha-Synuclein 111-132
ISIXEIXXVIVXXIETILFKKKKGILEDMPVDPDNEAYEMPSEEG

SEQ ID NO: 118 - protein, 30 aa, synthetic construct
  PEPTIDE 1..19: note = MvF5 Th
  SITE 20: note = epsilon-lysine as a spacer
  PEPTIDE 21..30: note = alpha-Synuclein 126-135
ISITEIKGVIVHRIETILFKEMPSEEGYQD

SEQ ID NO: 119 - protein, 42 aa, synthetic construct
  PEPTIDE 1..19: note = MvF5 Th
  SITE 20: note = epsilon-lysine as a spacer
  PEPTIDE 21..42: note = alpha-Synuclein 111-132
ISITEIKGVIVHRIETILFKGILEDMPVDPDNEAYEMPSEEG

SEQ ID NO: 120 - protein, 29 aa, synthetic construct
  PEPTIDE 1..18: note = HBsAg3 Th
  SITE 19: note = epsilon-lysine as a spacer
  PEPTIDE 20..29: note = alpha-Synuclein 126-135
KKKIITITRIITIITTIDKEMPSEEGYQD

SEQ ID NO: 121 - protein, 41 aa, synthetic construct
  PEPTIDE 1..18: note = HBsAg3 Th
  SITE 19: note = epsilon-lysine as a spacer
  PEPTIDE 20..41: note = alpha-Synuclein 111-132
KKKIITITRIITIITTIDKGILEDMPVDPDNEAYEMPSEEG

SEQ ID NO: 122 - protein, 40 aa, synthetic construct
  PEPTIDE 1..17: note = Clostridium tetani1 Th
  SITE 18: note = epsilon-lysine as a spacer
  PEPTIDE 19..40: note = alpha-Synuclein 111-132
KKQYIKANSKFIGITELKGILEDMPVDPDNEAYEMPSEEG

SEQ ID NO: 123 - protein, 38 aa, synthetic construct
  PEPTIDE 1..15: note = MvF1 Th
  SITE 16: note = epsilon-lysine as a spacer
  PEPTIDE 17..38: note = alpha-Synuclein 111-132
LSEIKGVIVHRLEGVKGILEDMPVDPDNEAYEMPSEEG

SEQ ID NO: 124 - protein, 47 aa, synthetic construct
  PEPTIDE 1..24: note = Bordetella pertussis Th
  SITE 25: note = epsilon-lysine as a spacer
  PEPTIDE 26..47: note = alpha-Synuclein 111-132
GAYARCPNGTRALTVAELRGNAELKGILEDMPVDPDNEAYEMPSEEG

SEQ ID NO: 125 - protein, 40 aa, synthetic construct
  PEPTIDE 1..17: note = Clostridium tetani2 Th
  SITE 18: note = epsilon-lysine as a spacer
  PEPTIDE 19..40: note = alpha-Synuclein 111-132
WVRDIIDDFTNESSQKTKGILEDMPVDPDNEAYEMPSEEG

SEQ ID NO: 126 - protein, 46 aa, synthetic construct
  PEPTIDE 1..23: note = Diphtheria Th
  SITE 24: note = epsilon-lysine as a spacer
  PEPTIDE 25..46: note = alpha-Synuclein 111-132
DSETADNLEKTVAALSILPGHGCKGILEDMPVDPDNEAYEMPSEEG

SEQ ID NO: 127 - protein, 44 aa, synthetic construct
  PEPTIDE 1..21: note = Plasmodium falciparum Th
  SITE 22: note = epsilon-lysine as a spacer
  PEPTIDE 23..44: note = alpha-Synuclein 111-132
DHEKKHAKMEKASSVFNVVNSKGILEDMPVDPDNEAYEMPSEEG

SEQ ID NO: 128 - protein, 40 aa, synthetic construct
  PEPTIDE 1..17: note = Schistosoma mansoni Th
  SITE 18: note = epsilon-lysine as a spacer
  PEPTIDE 19..40: note = alpha-Synuclein 111-132
KWFKTNAPNGVDEKHRHKGILEDMPVDPDNEAYEMPSEEG

SEQ ID NO: 129 - protein, 48 aa, synthetic construct
  PEPTIDE 1..25: note = Cholera Toxin Th
  SITE 26: note = epsilon-lysine as a spacer
  PEPTIDE 27..48: note = alpha-Synuclein 111-132
ALNIWDRFDVFCTLGATTGYLKGNSKGILEDMPVDPDNEAYEMPSEEG

SEQ ID NO: 130 - protein, 38 aa, synthetic construct
  PEPTIDE 1..15: note = MvF2 Th
  SITE 16: note = epsilon-lysine as a spacer
  PEPTIDE 17..38: note = alpha-Synuclein 111-132
ISEIKGVIVHKIEGIKGILEDMPVDPDNEAYEMPSEEG

SEQ ID NO: 131 - protein, 45 aa, synthetic construct
  VARIANT 7: note = serine or threonine
  VARIANT 10: note = lysine or arginine
  VARIANT 11: note = glycine or threonine
  VARIANT 15: note = histidine or threonine
  VARIANT 16: note = lysine or arginine
  VARIANT 19: note = glycine or threonine
  PEPTIDE 1..22: note = KKKMvF3 Th
  SITE 23: note = epsilon-lysine as a spacer
  PEPTIDE 24..45: note = alpha-Synuclein 111-132
KKKISIXEIXXVIVXXIEXILFKGILEDMPVDPDNEAYEMPSEEG

SEQ ID NO: 132 - protein, 41 aa, synthetic construct
  VARIANT 1: note = lysine or arginine
  VARIANT 2: note = lysine or arginine
  VARIANT 3: note = lysine or arginine
  VARIANT 4: note = leucine, isoleucine, valine or phenylalanine
  VARIANT 5: note = phenylalanine, lysine or arginine
  VARIANT 6: note = leucine, isoleucine, valine or phenylalanine
  VARIANT 7: note = leucine, isoleucine, valine or phenylalanine
  VARIANT 9: note = lysine or arginine
  VARIANT 10: note = leucine, isoleucine, valine or phenylalanine
  VARIANT 11: note = leucine, isoleucine, valine or phenylalanine
  VARIANT 13: note = leucine, isoleucine, valine or phenylalanine
  VARIANT 15: note = glutamine, leucine, isoleucine, valine or phenylalanine
  VARIANT 17: note = leucine, isoleucine, valine or phenylalanine
  VARIANT 18: note = aspartic acid or arginine
  PEPTIDE 1..18: note = HBsAg 1 Th
  SITE 19: note = epsilon-lysine as a spacer
  PEPTIDE 20..41: note = alpha-Synuclein 111-132
XXXXXXXTXXXTXPXSXXKGILEDMPVDPDNEAYEMPSEEG

SEQ ID NO: 133 - protein, 41 aa, synthetic construct
  VARIANT 4: note = isoleucine or phenylalanine
  VARIANT 5: note = isoleucine or phenylalanine
  VARIANT 6: note = threonine or leucine
  VARIANT 7: note = isoleucine or leucine
  VARIANT 11: note = isoleucine or leucine
  VARIANT 14: note = proline or isoleucine
  VARIANT 15: note = glutamine or threonine
  VARIANT 16: note = serine or threonine
  VARIANT 17: note = leucine or isoleucine
  PEPTIDE 1..18: note = HBsAg 2 Th
  SITE 19: note = epsilon-lysine as a spacer
  PEPTIDE 20..41: note = alpha-Synuclein 111-132
KKKXXXXTRIXTIXXXXDKGILEDMPVDPDNEAYEMPSEEG

SEQ ID NO: 134 - protein, 34 aa, synthetic construct
  PEPTIDE 1..11: note = Influenza MP1_1 Th
  SITE 12: note = epsilon-lysine as a spacer
  PEPTIDE 13..34: note = alpha-Synuclein 111-132
FVFTLTVPSERKGILEDMPVDPDNEAYEMPSEEG

SEQ ID NO: 135 - protein, 38 aa, synthetic construct
  PEPTIDE 1..15: note = Influenza MP1_2 Th
  SITE 16: note = epsilon-lysine as a spacer
  PEPTIDE 17..38: note = alpha-Synuclein 111-132
SGPLKAEIAQRLEDVKGILEDMPVDPDNEAYEMPSEEG

SEQ ID NO: 136 - protein, 32 aa, synthetic construct
  PEPTIDE 1..9: note = Influenza NSP1 Th
  SITE 10: note = epsilon-lysine as a spacer
  PEPTIDE 11..32: note = alpha-Synuclein 111-132
DRLRRDQKSKGILEDMPVDPDNEAYEMPSEEG

SEQ ID NO: 137 - protein, 42 aa, synthetic construct
  PEPTIDE 1..19: note = EBV BHRF1 Th
  SITE 20: note = epsilon-lysine as a spacer
  PEPTIDE 21..42: note = alpha-Synuclein 111-132
AGLTLSLLVICSYLFISRGKGILEDMPVDPDNEAYEMPSEEG

SEQ ID NO: 138 - protein, 38 aa, synthetic construct
  PEPTIDE 1..15: note = Clostridium tetani TT1 Th
  SITE 16: note = epsilon-lysine as a spacer
  PEPTIDE 17..38: note = alpha-Synuclein 111-132
QYIKANSKFIGITELKGILEDMPVDPDNEAYEMPSEEG

SEQ ID NO: 139 - protein, 43 aa, synthetic construct
  PEPTIDE 1..20: note = EBV EBNA-1 Th
  SITE 21: note = epsilon-lysine as a spacer
  PEPTIDE 22..43: note = alpha-Synuclein 111-132
PGPLRESIVCYFMVFLQTHIKGILEDMPVDPDNEAYEMPSEEG

SEQ ID NO: 140 - protein, 44 aa, synthetic construct
  PEPTIDE 1..21: note = Clostridium tetani TT2 Th
  SITE 22: note = epsilon-lysine as a spacer
  PEPTIDE 23..44: note = alpha-Synuclein 111-132
FNNFTVSFWLRVPKVSASHLEKGILEDMPVDPDNEAYEMPSEEG

SEQ ID NO: 141 - protein, 39 aa, synthetic construct
  PEPTIDE 1..16: note = Clostridium tetani TT3 Th
  SITE 17: note = epsilon-lysine as a spacer
  PEPTIDE 18..39: note = alpha-Synuclein 111-132
KFIIKRYTPNNEIDSFKGILEDMPVDPDNEAYEMPSEEG

SEQ ID NO: 142 - protein, 39 aa, synthetic construct
  PEPTIDE 1..16: note = Clostridium tetani TT4 Th
  SITE 17: note = epsilon-lysine as a spacer
  PEPTIDE 18..39: note = alpha-Synuclein 111-132
VSIDKFRIFCKALNPKKGILEDMPVDPDNEAYEMPSEEG

SEQ ID NO: 143 - protein, 41 aa, synthetic construct
  PEPTIDE 1..18: note = EBV CP Th
  SITE 19: note = epsilon-lysine as a spacer
  PEPTIDE 20..41: note = alpha-Synuclein 111-132
VPGLYSPCRAFFNKEELLKGILEDMPVDPDNEAYEMPSEEG

SEQ ID NO: 144 - protein, 37 aa, synthetic construct
  PEPTIDE 1..14: note = HCMV IE1 Th
  SITE 15: note = epsilon-lysine as a spacer
  PEPTIDE 16..37: note = alpha-Synuclein 111-132
DKREMWMACIKELHKGILEDMPVDPDNEAYEMPSEEG

SEQ ID NO: 145 - protein, 38 aa, synthetic construct
  PEPTIDE 1..15: note = EBV GP340 Th
  SITE 16: note = epsilon-lysine as a spacer
  PEPTIDE 17..38: note = alpha-Synuclein 111-132
TGHGARTSTEPTTDYKGILEDMPVDPDNEAYEMPSEEG

SEQ ID NO: 146 - protein, 36 aa, synthetic construct
  PEPTIDE 1..13: note = EBV BPLF1 Th
  SITE 14: note = epsilon-lysine as a spacer
  PEPTIDE 15..36: note = alpha-Synuclein 111-132
KELKRQYEKKLRQKGILEDMPVDPDNEAYEMPSEEG

SEQ ID NO: 147 - protein, 34 aa, synthetic construct
  PEPTIDE 1..11: note = EBV EBNA-2 Th
  SITE 12: note = epsilon-lysine as a spacer
  PEPTIDE 13..34: note = alpha-Synuclein 111-132
TVFYNIPPMPLKGILEDMPVDPDNEAYEMPSEEG

SEQ ID NO: 148 - protein, 4 aa, synthetic construct
  SITE 1: note = epsilon-lysine
  PEPTIDE 1..4: note = epsilon lysine - lysine-lysine-lysine as a spacer
KKKK

SEQ ID NO: 149 - protein, 6 aa, synthetic construct
  VARIANT 3: note = Any amino acid
  VARIANT 5: note = Any amino acid
PPXPXP